(19) United States
(12) Patent Application Publication
(10) Pub. No.: US 2011/0060000 A1
(43) Pub. Date: Mar. 10, 2011

(54) ACRIDINE ANALOGS IN THE TREATMENT OF GLIOMAS

(76) Inventors: Maurizio Grimaldi, Birmingham, AL (US); Subramaniam Ananthan, Birmingham, AL (US)

(21) Appl. No.: 12/879,464

(22) Filed: Sep. 10, 2010

Related U.S. Application Data
(60) Provisional application No. 61/241,168, filed on Sep. 10, 2009.

(51) Int. Cl. A61K 31/473; A61P 35/00; A61P 35/02; A61K 31/4706

(52) U.S. Cl. 514/297; 514/313

(57) ABSTRACT

Disclosed are methods and compositions for treating gliomas that involve quinacrine and other acridine analogs. This abstract is intended as a scanning tool for purposes of searching in the particular art and is not intended to be limiting of the present invention.

### CROSS-REFERENCE TO RELATED APPLICATIONS

This application claims the benefit of U.S. Application No. 61/241,168, filed Sep. 10, 2009; which is hereby incorporated herein by reference in entirety.

### BACKGROUND

Gliomas represent an unmet medical need. Gliomas are the most frequent primary adult neoplasm originating from brain tissues. They usually generate from the glial lineage. Brain tumors represent 1.3% of all the cancers and 2.2% of all the cancer related deaths. It is established that brain cancers cause about 22,000 deaths annually in the USA (American cancer society epidemiologic data).

Astrocytomas are the most frequent primary brain tumors (35%) in the adult. They can be classified in three grades; low grade, intermediate grade, and high grade astrocytoma. The hallmark of these cancers is the impossibility to perform a radical surgical resection given their ability to grow within the noble brain parenchyma and their close proximity with vital brain centers. Although astrocytomas are not able to cause distant organ metastasis, they can relapse at different brain locations than their primary lesion. Hence, they are believed to be multifocal. Other theories indicate that the cancer cells, of clonal origin, can spread early through the brain and secondary tumors appear, symptomatically, with different timing. Astrocytomas are resistant to chemio- and radio-therapy and are invariably lethal. Low grade astrocytomas have a survival rate at 5 years of 25% in adult over 45. The 5 year survival rate for high grade astrocytoma is 2% or less. It is clear how lethal these tumors are and that there is a need for more research in this field.

Preliminary studies have indicated that the edoplasmic reticulum stress response (ERSR) can be a target in glioma management (Kardosh et al., 2007, “Reduced survivin expression and tumor cell survival during chronic hypoxia and further cytotoxic enhancement by the cyclooxygenase-2 inhibitor celecoxib.” J Biomed Sci 14(5): 647-62; Pyrko et al., 2007, “Calcium-activated endoplasmic reticulum stress as a major component of tumor cell death induced by 2,5-dimethyl-celecoxib, a non-coxib analogue of celecoxib,” Mol Cancer Ther 6(4): 1262-75; Pyrko et al., 2008, “Celecoxib transiently inhibits cellular protein synthesis,” Biochem Pharmacol 75(2): 395-404; Pyrko et al., 2007, “HIV-1 protease inhibitors nelfinavir and atazanavir induce malignant glioma death by triggering endoplasmic reticulum stress,” Cancer Res 67(22): 10920-8). Briefly, the endoplasmic reticulum (ER) is able to serve a large variety of roles (Estrada de Martin et al., 2005, “The organization, structure, and inheritance of the ER in higher and lower eukaryotes,” Biochem Cell Biol 83(6): 752-61). For example, proteins can be synthesized on its surface and in the presence of the leader signal, they are internalized in the ER to be further processed, assembled, packed and stored or targeted to their final location. Upon request, stored ER proteins can be accessed, sorted, and shipped to the final cellular location with admirable precision. Protein storage in the ER is achieved with the use of several molecular and ionic chaperones, which direct proper protein folding to achieve high efficiency; high density packing takes up less space. The ER is not only a protein assembly, storage, and shipping facility, but it is also a Ca²⁺ storage organelle, which participates in a number of key signaling processes. High quantity of Ca²⁺ cannot be stored in the normal cellular environment due to low solubility in a phosphate environment. To work around this problem, proteins to be stored in the ER are used to buffer Ca²⁺ and thus prevent its precipitation. In fact, proteins to be stored in the ER are packed in a very space efficient manner, using Ca²⁺ as an ionic chaperon or nucleating factor (Michalak et al., 2002, “Ca2+ signaling and calcium binding chaperones of the endoplasmic reticulum,” Cell Calcium 32(5-6): 269-78). In this way, higher quantity of Ca²⁺ can be kept in the ER without precipitating and can be released quite quickly for signaling purposes, as the ER pool of free Ca²⁺ is released in the cytoplasm. In this configuration Ca²⁺ can be stored in the ER at concentrations up to 100 μM. A similar process of Ca²⁺ accumulation occurs in the mitochondria. Differently from the ER, Ca²⁺ in this organelle is stored at higher concentrations and quantities, usually mMoles, as crystals. Mitochondria are a significantly slower Ca²⁺ reservoir, and are involved in different Ca²⁺ buffering and releasing processes. In normal conditions, depletion of Ca²⁺ in the ER occurs quickly, and then the ion is pumped back in the ER by the Smooth ER Ca²⁺ ATPase (SERCA), so that it is replenished as soon as possible. However, if Ca²⁺ depletion is prolonged and profound, ER stored proteins can start to unfold (Yoshida et al., 2006, “Depletion of intracellular Ca²⁺ store itself may be a major factor in thapsigargin-induced ER stress and apoptosis in PC12 cells,” Neurochem Int 48(8): 696-702). Also, newly synthesized proteins can not fold properly in these low Ca²⁺ conditions, also because of the failure of chaperones due to their dependence on Ca²⁺. Unfolding of the proteins, in turn, triggers a reaction in the cells called ER stress response (ERSR), which can ultimately lead to cell death via apoptosis (Soboloff et al., 2002, “Sustained ER Ca2+ depletion suppresses protein synthesis and induces activation-enhanced cell death in mast cells,” J Biol Chem 277(16): 13812-20). ERSR can also be triggered by other means. For example, the impossibility of the protein in the ER to be folded for the lack of N- and O-glycosylation triggers ERSR. This latter pathway was initially described as the unfolded protein response (UPR). Although, triggered by different mechanisms, UPR and ERSR share the same downstream mechanisms. Additionally, ERSR can be triggered by failure of the proteasomal-mediated damaged protein removal.

Although preliminary studies and the data present in the literature have indicated that ERSR can be a target in glioma management (Kardosh et al., 2007; Pyrko et al., 2008; Pyrko et al., 2007), and that the results obtained translate in effectiveness in xenograft models, there are several and severe limitations to these earlier attempts. These limitations include poor brain bioavailability in the case of HIV-PIs and severe and life-threatening side effects in the case of COX2 inhibitors. In addition, not enough information is available to understand the role of these compounds.

What are desperately needed are new compounds and methods for treating gliomas. Disclosed herein are compositions and methods related to the use of various small molecules, e.g., quinacrine and other acridine analogs, as an agent to be used alone, in combination with each other and/or in combination with other antineoplastic drugs for the treatment of gliomas through direct tumor toxicity, inhibition of cancer cell proliferation, prevention of readhesion and relapse and also to be used intraoperativelly as a bathing solution to prevent spreading during surgical removal of the primary tumor.

### SUMMARY

In accordance with the purposes of the disclosed materials, compounds, compositions, articles, and methods, as embodied and broadly described herein, the disclosed subject matter, in one aspect, relates to compositions and methods for preparing and using such compositions. In a further aspect, the disclosed subject matter relates to methods of using quinacrine and other acridine analogs, used alone or in combination, to treat gliomas and methods of using quinacrine and other acrine compounds intraoperativelly as a bathing solution to prevent spreading during surgical procedures.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a graph that shows that intracellular Ca²⁺ stores are quickly and efficiently refilled after CCE initiation even in the absence of [Ca²⁺]ᵢ elevation. Astrocytes were perfused with Ca²⁺-free extracellular solution and exposed to 10 μM ATP. The agonist was removed and cells were perfused with an extracellular solution containing 1 mM Ca²⁺ for 60 sec. Then ATP was reintroduced to test the ability of the ICS to release Ca²⁺. (Perfusion of all applied substances is indicated by the horizontal bars.) (Grimaldi, 2006, “Astrocytes refill intracellular Ca²⁺ stores in the absence of cytoplasmic [Ca²⁺] elevation: a functional rather than a structural ability,” J Neurosci Res 84(8): 1738-49.).

FIG. 2 shows a graph that shows the effect of Thapsigargin (THAP) on Astrocytes and C6. Exposure of astrocytes and C6 to 2 μM THAP caused elevation of [Ca²⁺]ᵢ. However, unexpectedly, the elevation of [Ca²⁺]ᵢ in C6 was significantly higher than in astrocytes. (Statistical analysis it is shown in the inset. Values at the peak of the response have been analyzed using ANOVA followed by T-test, ** indicate P<0.01.).

FIG. 3 shows a graph that shows the effect of ATP on [Ca²⁺]ᵢ in astrocytes and C6. Ca²⁺ mobilization from ER was caused using an agonist acting via inositol trisphosphate elevation, mimicking physiological responses. In these conditions the dynamics of Ca²⁺ transients were observe in these two cell lines. It appears evident that the plateau phase in C6 is consistently larger than in astrocytes.

FIG. 4 shows a graph that shows the effect of THAP in astrocytes and C6 in the absence of extracellular Ca²⁺. THAP causes release of calcium from ER. There is no difference in the amount of Ca²⁺ stored and released from the ER between astrocytes and C6. This indicates that the amount of Ca²⁺ stored in the ER is about the same in these two cell types. N.S. Indicate the lack of statistical difference between the two cell types.

FIG. 5 shows a graph that shows extracellular Ca²⁺ influx following THAP-induced ER Ca²⁺ depletion in astrocytes and C6. ER has been discharged of Ca²⁺ using a Ca²⁺-free KRB and THAP as shown in FIG. 4. After reaching baseline, extracellular Ca²⁺ was re-added to initiate capacitative influx. There were no apparent differences in the two cell types indicating that Ca²⁺ influx is not different in these cell types.

FIG. 6 shows a graph that shows the effect of ATP on Ca²⁺ mobilization in astrocytes and C6 in the absence of extracellular Ca²⁺ and following influx. Ca²⁺ mobilization from ER was caused using an agonist acting via inositol trisphosphate elevation in the absence of extracellular Ca²⁺. In these conditions, the dynamics of calcium transients were observed in these two cell lines. It appears evident that the plateau phase in C6 is consistently larger than in astrocytes. In these conditions SERCA is mostly responsible for shaping these transients in astrocytes. Most likely SERCA in these cells is not able to handle the same amount of Ca²⁺ than in astrocytes. At the end of the experiment, reintroduction of Ca²⁺ initiates capacitative influx that is similar in the two cell lines.

FIG. 7 shows a pair of graphs that show the effect of Tunicamycin (TUN) pretreatment in astrocytes and C6. Panel A: Challenge of astrocytes with ATP show the unchanged typical spike and plateau response. Panel B: In C6 an oscillatory pattern is readily detectable in response to treatment with TUN.

FIG. 8 shows SERCA expression in astrocytes and C6. Astrocytes and C6 were grown and proteins were purified by immunoprecipitation using a SERCA antibody. Immunoprecipitated proteins were separated by SDS-page and immunoblotted with a second SERCA antibody. A single band was identified at 127 KDa corresponding to SERCA molecular weight (Panel A). The identified band was significantly larger in astrocytes than in C6 (Panel B). ** P>0.01 astrocytes versus C6.

FIG. 9 shows concentration related effects of THAP on the expression of GRP78. Astrocytes and C6 were exposed to graded concentration of THAP as indicated in the captions for 24 hours. Thereafter, protein were extracted and run on a SDS-PAGE and immunoblotted with an anti-GRP78 antibody. Filters were exposed to ECL and films were impressed and developed (Panel B). Films were digitalized using a high resolution scanner and analyzed used imageJ (NIH). Densitometric values were averaged and plotted in the graph (Panel A). Loading conditions were checked using GAPDH housekeeping gene. GRP 78 showed a stronger elevation in response to THAP in C6 as compared to astrocytes.

FIG. 10 shows the effect of TUN on GRP78 protein expression. Astrocytes and C6 were exposed to TUN for 8 hours at concentrations ranging from 1.25 μg/ml to 10 μg/ml. Proteins were extracted, separated on a SDS-PAGE and immunoblotted. Filters were exposed to ECL and used to impress films (Panel B). Developed films were subsequently digitized with a high resolution scanner and analyzed with the software ImageJ (NIH). Densitometric values were averaged and plotted in the graph (Panel A).

FIG. 11 shows GRP78 expression in astrocytes, C6, and U78-MG in resting conditions and after challenge with THAP and TUN. Cells were treated as specified in the graph labeling. It is evident that glioma cells have enhanced GRP78 expression than astrocytes regardless of the challenging agent. N.T.=untreated; D=DMSO; T=TUN; Th=THAP.

FIG. 12 shows a graph that shows the effect of ERSR induction on cell viability. Cells were treated for 48 hours accordingly to axis labels. At the end of the incubation time cells were prepared for the assay according to the manufacturer instructions. Luminescence values were then converted to % of survival. Both C6 and U87-MG were significantly more sensitive to killing by ERSR induction than primary astrocytes. ** indicates p<0.01 vs. respective DMSO control; ▪ ▪ and vs. same treatment astrocytes.

FIG. 13 shows a graph that shows the effect of THAP and TUN on cells survival as assessed by level of the cytoplasmic exosaminidase. Cells were treated for 48 h with the indicated concentrations of the agents. Astrocytes death was limited to 30% or lower. Suprisingly, C6 death was 3 folds higher than in astrocytes at 24 h and increased at 5 folds at 48 h. Data in U78-MG are similar to the C6 data although the effect was slightly lower but always at least 2 to 4 folds the effect seen in astrocytes. Statistical validation performed by ANOVA followed by the ad hoc T-Test showed significant differences in the death of C6 and U87-MG compared to primary astrocytes subjected to the same treatment.

FIG. 14 shows the effect of ERSR activation in astrocytes and C6 on procaspase cleavage and release of caspase 12 active form. Cells were treated with the agents as indicated in the x axis. Subsequently cells were harvested and protein was extracted and then separated via SDS-PAGE and immunoblotted. The arrow highlights the 45 kDa band that results from cleavage of procaspase 12 and corresponding to the active caspase.

FIG. 15 shows the effect of Thap and staurosporin (STS) on TUNEL labeling. C6 were plated on glass coverslips. Cells were fixed and stained accordingly to manufacturer instructions. Propidium staining of the nuclei was replaced by Hoechst included in the mounting media. Several images were acquired with a high resolution camera on a microscope equipped with a 10× long working distance dry lens and analyzed with powerful commercial software. Results were expressed in the graph as green labeled pixel counts. The images from the top to the bottom are respectively from N.T., DMSO, Thap 20 nM, Thap 200 nM, STS100 nM. The blue images reflect nuclear staining while the images on the right represent BRDU staining.

FIG. 16 shows a schematic representation of GRP78-luc reporter. The promoter region contains the sequence of the GRP78 promoter from −139 to +7. In this part of the promoter there are three ER stress responsive elements and the TATA box to which is linked in frame the firefly luciferase sequence.

FIG. 17 shows a graph of luciferase expression in stably transfected U87-MG. Baseline and induced expression of luciferase in U87-MG transduced with the pGRP78-luc construct. Robust expression of luciferase signal induced by positive controls was observed after 24 h incubation period. In particular, a selected clone A5 showed a 6-7 fold induction upon exposure to low THAP and TUN concentrations.

FIG. 18 shows a graph of the effect of incubation time on luciferase induction by positive controls in A5. 8 h exposure to positive controls resulted in a very small induction of luciferase. 16 h exposure resulted in the highest signal. 24 and 38 h showed a significantly lower induction due to the toxic effect of the compounds.

FIG. 19 shows a graph of minimum cell density assessment. The cell dependency of the assay was tittered in half area 96 wells. In this assay a range of cells between 4 and 8 thousands performs well enough in terms of dynamic range and the folds of induction is not affected by reducing the cell number in this range.

FIG. 20 shows two examples of inverted quadrant Z-plate arrays. Z-number assessments were performed using 2 positive controls THAP and TUN. As it can be appreciated z-values are significantly high and above 0.65 on a reproducible basis with both positive controls. This indicates the solidity of the assay.

FIG. 21 shows a pair of graphs profiling the effect of THAP and TUN in A5. Both TUN and THAP elicited a concentration dependent increase of grp78 promoter driven luciferase. The profile of the two agents overlaps the effect on GRP78 protein expression and their known pharmacological profile. This indicates that the construct is expressed in the engineered cell line in accordance with its predicted biological properties.

FIG. 22 shows examples of a plate layout deriving from a semi-automated screening of the Prestwick library. Compounds were plated in half area 96 well plates at the final concentration of 2 μM in 5 μl of media. Cells were added in 45 μl afterwards at the density of 5000/well. Plates were read after 16 h in an Envision high efficiency plate reader. Results are represented as a plate layout in which different shadings indicate the different rows and the number indicate the different lines. As a standard negative and positive controls were located in the outer columns.

FIG. 23 shows a graph showing the selection of the hits. It was determined that values 3 S.D. above the negative controls correlates with high probability effective compounds. The twelve compounds shown satisfied these criteria.

FIG. 24 shows a group of graphs that show the effect of quinacrine on GRP78-Luc expression and gliotoxicity. Quinacrine increased expression of GRP78-luc up to 10 μM after which the toxic effect was preponderant (panel A). Parallely quinacrine in the same concentration range killed very effectively both C6 (graph B) and U87-MG cells (graph C).

FIG. 25 shows the comparison between different vendor quinacrines (the chemical structure is provided). In particular quinacrine provided from Sigma at low cost show a little less effect than quinacrine provided by other vendors. However, after adjustment of the solubilization procedure these differences were decrease.

FIG. 26 shows that OSSL 53454 (3-chloro-N⁹-(5-(diethlyamino)pentan-2-yl)-7-methoxy acridine-2,9-diamine) causes an increase of the GRP78-luc reporter gene as quinacrine. Analogoulsy this compound also causes gliotoxicity as with quinacrine.

FIG. 27 shows that 9-aminoacridine, hydrate, hydrochloride similarly causes increased expression of the GRP78-luc reporter gene and also causes gliotoxicity.

FIG. 28 shows a graph of the effect of 200 mg/kg of quinacrine after daily oral administration for only 14 days in mice bearing subcutaneous U87-MG gliomas. In the figure it is shown that control animal tumors grow at a high rate, while in quinacrine treated animals the tumors decrease in size and even after withdrawal of the treatment their size is greatly smaller than in control animals. One of the treated animals in this group showed disappearance of the tumor. The estimated survival prolongment is 14 days in mice life which compares at 851 days in human life based on a life expectancy of 18 months in mice and 90 years in human. Also, since one animal was tumor-free at the end of the treatment that translates in an expected 16% cure rate.

FIG. 29 shows the effect of quinacridine (Q) and 9-aminoacridine (9-AA) in in vivo glioma models for U87-MG skin xenograft. FIG. 29a graphs % tumor weight/control as a function of days after implantation. Oral treatment with Quinacridine (Q) and I.P. administration of 9-aminoacridine (9-AA) each resulted in a decrease in tumor size, compared to control, with full dose over a period of about 45 days. Afterwards, a half dose (maintenance dose) was employed up to about 60 days; tumor size remained relatively constant, compared to control, for both quinacridine (Q) and 9-aminoacridine (9-AA). Such a profile indicates effectiveness in vivo for both treatment and for prevention of relapse. FIG. 29b graphs % animal survival as a function of days after implantation. Oral treatment with quinacridine (Q) and I.P. administration of 9-aminoacridine (9-AA) each resulted in increased survival rate, compared to control, with full dose over a period of about 45 days. Afterwards, a half dose (maintenance dose) was employed up to about 60 days; survival rate was high, compared to control, for both quinacridine (Q) and 9-aminoacridine (9-AA). With 9-aminoacridine (9-AA), there was no sign of tumor once survival rate stabilized. Such a profile indicates effectiveness in vivo for both treatment and for prevention of relapse.

FIG. 30 shows the effect of quinacridine (Q) and 9-aminoacridine (9-AA) in in vivo glioma models for U87-MG skin xenograft tumor explants. U7 represents vehicle treated sample after 7 days. U21 represents vehicle treated sample after 21 days. Q7 represents quinacridine-treated sample after 7 days. Q21 represents quinacridine-treated sample after 21 days. 9AA-7 represents 9-aminoacridine-treated sample after 7 days. 9AA-21 represents 9-aminoacridine-treated sample after 21 days. In the figure it is clearly evident the activaiton of ERSR and changes in GRP78 target protein. Also, activation of caspase 12 is evident.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. GENERAL DEFINITIONS

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such component, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

“Optional” or “optionally” means that the subsequently described event or circumstance may or may not occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment prior to the administering step. In some aspects of the disclosed methods, the subject has been identified to be in need of treatment for a disorder, which refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

### B. CHEMICAL DEFINITIONS

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, amino, ester, ether, halide, hydroxy or ketone, as described below. The term “lower alkyl” means an alkyl group of from 1 to 6 carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OA¹ where A¹ is alkyl as defined above.

The term “alkoxylalkyl” as used herein is an alkyl group that contains an alkoxy substituent and can be defined as -A¹-O-A², where A¹ and A² are alkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, amino, ester, ether, halide, hydroxy or ketone, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, amino, ester, ether, halide, hydroxyor ketone, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, amino, ester, ether, halide, hydroxy or ketone, as described herein. The term “biaryl” is a specific type of aryl group and is included in the definition of aryl. Biaryl refers to two aryl groups that are bound together via a fused ring structure, as in naphthalene or quinaline, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, amino, ester, ether, halide, hydroxy or ketone, as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, amino, ester, ether, halide, hydroxy or ketone, as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above. Throughout this specification “C(O)” is a short hand notation for C═O.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{O}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched)alkylene)O—N(R^{∘}₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{*}₂, ═NNHC(O)R*, ═NNHC(O)OR*, ═NNHS(O)₂R*, ═NR*, ═NOR*, —O(C(R^{*}₂))₂₋₃O—, or —S(C(R^{*}₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

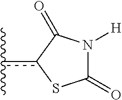

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable minor images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

In some aspects, a structure of a compound can be represented by a formula:

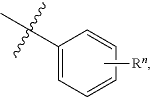

which is understood to be equivalent to a formula:

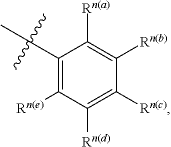

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

### C. BIOLOGICAL DEFINITIONS

It is understood that one way to define any known variants and compounds or those that might arise, of the disclosed genes and proteins herein is through defining the variants and compounds in terms of homology to specific known sequences. For example, the sequences of a particular human GRP78 or SERCA are known and readily obtainable at for example, a sequence database such as Genbank. The nucleic acids that encode these proteins are also readily available. These sequences and any known alleles or species variants are considered disclosed herein. Specifically disclosed are variants of these and other genes and proteins herein disclosed which have at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman, Adv Appl Math 2:482, 1981, by the homology alignment algorithm of Needleman and Wunsch, J Mol. Biol. 48:443, 1970, by the search for similarity method of Pearson and Lipman, Proc Natl Acad Sci USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, Science 244:48-52, 1989, Jaeger et al., Proc Natl Acad Sci USA 86:7706-7710, 1989, Jaeger et al., Methods Enzymol 183:281-306, 1989, which are herein incorporated by reference for at least material related to nucleic acid alignment.

There are a variety of molecules disclosed herein that are nucleic acid based, including for example the nucleic acids that encode, for example GRP78, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell that the expressed mRNA will typically be made up of A, C, G, and U Likewise, it is understood that if, for example, an antisense molecule is introduced into a cell or cell environment through for example exogenous delivery it is advantageous that the antisense molecule be made up of nucleotide analogs that reduce the degradation of the antisense molecule in the cellular environment.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc Natl Acad Sci USA, 86:6553-6, 1989).

There are a variety of sequences related to for example, the GRP78 genes, found in sequence data bases, such as Genbank. These sequences and others are herein incorporated by reference in their entireties as well as for individual subsequences contained therein.

Those of skill in the art understand how to resolve sequence discrepancies and differences and to adjust the compositions and methods relating to a particular sequence to other related sequences (i.e., sequences of GRP78). Primers and/or probes can be designed for any GRP78 sequence given the information disclosed herein and known in the art.

Disclosed are compositions including primers and probes, which are capable of interacting with, for example, the GRP78 nucleic acids, such as mRNA, as disclosed herein. In certain examples the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain examples the primers are used for the DNA amplification reactions, such as PCR or direct sequencing. It is understood that in certain examples the primers can also be extended using non-enzymatic techniques, where for example, the nucleotides or oligonucleotides used to extend the primer are modified such that they will chemically react to extend the primer in a sequence specific manner. Typically the disclosed primers hybridize with, for example, the GRP78 nucleic acid, such as mRNA, or region of the GRP78 nucleic acids or they hybridize with the complement of the GRP78 nucleic acids or complement of a region of the GRP78 nucleic acids.

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of GRP78 or the genomic DNA of GRP78 or they can interact with the polypeptide of GRP78. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place. Both of these recognition motifs can also occur in the same functional nucleic acid molecule.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than 10⁻⁶. It is more preferred that antisense molecules bind with a k_{d} less than 10⁻⁸. It is also more preferred that the antisense molecules bind the target molecule with a k_{d} less than 10⁻¹⁰. It is also preferred that the antisense molecules bind the target molecule with a k_{d} less than 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶. It is more preferred that the aptamers bind the target molecule with a k_{d} less than 10⁻⁸. It is also more preferred that the aptamers bind the target molecule with a k_{d} less than 10⁻¹⁰. It is also preferred that the aptamers bind the target molecule with a k_{d} less than 10⁻¹². Aptamers can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). It is preferred that the aptamer have a k_{d} with the target molecule at least 10 fold lower than the k_{d} with a background binding molecule. It is more preferred that the aptamer have a k_{d} with the target molecule at least 100 fold lower than the k_{d} with a background binding molecule. It is more preferred that the aptamer have a k_{d} with the target molecule at least 1000 fold lower than the k_{d} with a background binding molecule. It is preferred that the aptamer have a k_{d} with the target molecule at least 10000 fold lower than the k_{d} with a background binding molecule. It is preferred when doing the comparison for a polypeptide for example, that the background molecule be a different polypeptide. For example, when determining the specificity of GRP78 aptamers, the background protein can be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶. It is more preferred that the triplex forming molecules bind with a k_{d} less than 10⁻⁸. It is also more preferred that the triplex forming molecules bind the target molecule with a k_{d} less than 10⁻¹⁰. It is also preferred that the triplex forming molecules bind the target molecule with a k_{d} less than 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc Natl Acad Sci USA 89:8006-10, 1992; WO 93/22434 by Yale; WO 95/24489 by Yale; Yuan and Altman, EMBO J. 14:159-68, 1995, and Carrara et al., Proc Natl Acad Sci USA 92:2627-31, 1995). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162

### D. DELIVERY OF THE COMPOSITIONS TO CELLS

### 1. Nucleic Acid Delivery

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, et al., Science, 247, 1465-1468, 1990; and Wolff, Nature, 352, 815-818, 1991. Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

In the methods described herein, which include the administration and uptake of exogenous DNA into the cells of a subject (i.e., gene transduction or transfection), the nucleic acids can be in the form of naked DNA or RNA, or the nucleic acids can be in a vector for delivering the nucleic acids to the cells, whereby the encoding DNA or DNA or fragment is under the transcriptional regulation of a promoter, as would be well understood by one of ordinary skill in the art as well as enhancers. The vector can be a commercially available preparation, such as an adenovirus vector (Quantum Biotechnologies, Inc. (Laval, Quebec, Canada).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof) disclosed herein. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al, Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed subject matter can be used in conjunction with any of these or other commonly used gene transfer methods.

As one example, if the antibody-encoding nucleic acid or some other nucleic acid encoding an inhibitor of the GRP78 protein or encoding a particular variant of the GRP78 gene to be used in the disclosed methods, is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can become integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### 2. Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed compositions or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

### 3. In Vivo/Ex Vivo

As described above, the compositions can be administered in a pharmaceutically acceptable carrier and can be delivered to the subjects cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### 5. Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. β actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins et al., Proc. Natl. Acad. Sci. 78:993, 1981) or 3′ (Lusky et al., Mol. Cell. Bio. 3:1108, 1983) to the transcription unit. Furthermore, enhancers can be within an intron (Banerji et al., Cell 33:729, 1983) as well as within the coding sequence itself (Osborne et al., Mol. Cell. Bio. 4:1293, 1984). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain examples the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### 6. Markers

The viral vectors can include nucleic acid sequence encoding a marker product. This marker product is used to determine if the gene has been delivered to the cell and once delivered is being expressed. Preferred marker genes are the E. coli lacZ gene, which encodes β-galactosidase, and green fluorescent protein.

In some examples the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg, 1982, J Molec Appl Genet 1:327), mycophenolic acid, (Mulligan and Berg, 1980, Science 209:1422) or hygromycin, (Sugden et al., 1985, Mol Cell Biol 5:410-413). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### E. SERCA (SMOOTH ER CA²⁺ ATPASE)

In most animal cells and plant cells, the normal concentration of free cytosolic Ca²⁺ is 50 to 100 nM. Since Ca²⁺ acts as a major intracellular messenger, elevating these levels affects a wide range of cellular processes including contraction, secretion and cell cycling (Dawson, 1990, Essays Biochem. 25:1-37; Evans et al., 1991, J. Exp. Botany 42:285-303). Intracellular Ca²⁺ stores hold a key position in the intracellular signaling. They allow the rapid establishment of Ca²⁺ gradients, and accumulate and release Ca²⁺ in order to control cytosolic Ca²⁺ levels. Moreover, lumenal Ca²⁺ intervenes in the regulation of the synthesis, folding and sorting of proteins in the endoplasmic reticulum (Brostrom and Brostrom, 1990, Ann. Rev. Physiol. 52:577-590; Suzuki et al., 1991, J. Cell. Biol. 114:189-205; Wileman et al., 1991, J. Biol. Chem. 266:4500-4507). Furthermore, it controls signal-mediated and passive diffusion through the nuclear pore (Greber and Gerace, 1995, J. Cell. Biol. 128:5-14).

Three genes that code for five different isoforms of the sarco/endoplasmic reticulum Ca²⁺ATPase (SERCA) are known in vertebrates, SERCAla/b, SERCA2a/b and SERCA3. The SERCA isoforms are usually tagged to the endoplasmic reticulum (ER) or ER subdomains like the sarcoplasmic reticulum, although the precise subcellular location is often not known. The SERCA proteins belong to the group of ATP-driven ion-motive ATPases, which also includes, amongst others, the plasma membrane Ca²⁺-transport ATPases (PMCA), the Na+-K+-ATPases, and the gastric H+-K+-ATPases. The SERCA Ca²⁺-transport ATPases can be distinguished from their plasma membrane counterparts like PMCA by the specific SERCA inhibitors: thapsigargin, cyclopiazonic acid, and 2,5-di(tert-butyl)-1,4-benzohydroquinone (Thastrup et al., 1990, PNAS USA 87:2466-2477; Seidler et al., 1989, J. Biol. Chem. 264:17816-17823; Oldershaw and Taylor, 1990, FEBS Lett. 274:214-216). In view of the diverse role of Ca²⁺ in the cell and the fact that Ca²⁺ is stored in diverse organelles, the diversity in Ca²⁺-accumulation pump isoforms is not surprising.

SERCA inhibitors have been described by Thomas et al. In: A Practical Guide to the Study of Calcium in Living Cells (Meth. Cell Biol., 40), Academic Press, San Diego, pp 65-89 (1994). The three most commonly used SERCA inhibitors are thapsigargin (Thastrup et al., 1990; and Lytton et al., 1991, J. Biol. Chem., 266:17067-17071), cyclopiazonic acid (Goeger et al., 1989, Biochem. Pharmacol., 38:3995-4003) and DBHQ (Moore et al., 1987, FEBS Lett., 224:331-336; and Kass et al., 1989, J. Biol. Chem., 264:15192-15198). Other SERCA inhibitors include pesticides or basic compounds for the development of pesticides such as herbicides, insecticides, and nematocides.

### F. ERSR (ENDOPLASMIC RETICULUM STRESS RESPONSE)

Regardless of how protein unfolding in the ER is initially triggered, the presence of unfolded protein in the ER begins a cascade of events aimed to deal with the accumulation of non-functional proteins. The clue to the process is the enhancement of the expression of several molecular chaperones, whose role, amongst many others, is to enhance the cells' ability to promote protein folding or to target them to proteasome-mediated degradation (for A review see Li et al., 2006, “Stress induction of GRP78/BiP and its role in cancer, Curr Mol Med 6(1): 45-54).

The transcription of several molecular elements is triggered upon the appearance of unfolded proteins in the ER (for a review see Oyadomari et al., 2004, “Roles of CHOP/GADD153 in endoplasmic reticulum stress,” Cell Death Differ 11(4): 381-9.). They can be grouped in two different classes: the chaperones which directly and indirectly help protein folding and the non chaperones. The primary responder to ERSR is the chaperone GRP78. The GRP78 gene promoter has several ER sensitive elements (ERSE) but also non ERSE activated regulation regions (Yoshida et al., 1998, “Identification of the cis-acting endoplasmic reticulum stress response element responsible for transcriptional induction of mammalian glucose-regulated proteins. Involvement of basic leucine zipper transcription factors, J Biol Chem 273(50): 33741-9). These elements are bound by several transcription factors, including TFII-I, YY1, SP1, NF-Y and the nuclear form of ATF6. Of all these transcription enhancers, ATF6 is the most powerful inducer of ERSE driven GRP78 transcription (Shen et al., 2002, “ER stress regulation of ATF6 localization by dissociation of BiP/GRP78 binding and unmasking of Golgi localization signals,” Dev Cell 3(1): 99-111). In the absence of unfolded proteins GRP78 binds to different proteins such as IRE1-a, PERK and ATF6. GRP78-bound IRE1-a, PERK and ATF6 are inactive (for review see Li et al., 2006; Oyadomari et al., 2004). Upon appearance of unfolded proteins in the ER, GRP78 binds the unfolded proteins and these three factors are released. ATF6 translocates to the Golgi, where it is cleaved by S1P/S2P to release its active form. Cleaved ATF6 translocates to the nuclei, form a complex with NF-Y bound to ERSE, and activates GRP78/BiP transcription. Cleaved ATF6 activation state can be enhanced by post-translational modifications such as phosphorylation by p38MAP kinase. Non ERSE activated transcription of GRP78 is achieved via ATF4-ATF1 and CREB1 which all bind upstream of ERSE to activate transcription. Similar to GRP78, GRP94, another chaperone, is also up-regulated. In addition to these well known chaperones, other proteins that help with post-translational maturation, such as protein disulfide isomerase (PDI) are also upregulated. Finally, structural ER proteins such as the Smooth ER Ca²⁺ ATPase (SERCA) are also up regulated during ERSR (Hojmann et al., 2001, “Upregulation of the SERCA-type Ca2+ pump activity in response to endoplasmic reticulum stress in PC12 cells, BMC Biochem 2: 4). In fact, SERCA by increasing the uptake of Ca²⁺ in the ER can improve protein folding since Ca²⁺ acts as a nucleation factor, or ionic chaperone and by activating Ca²⁺-dependent chaperones. If ERSR is not able to deal with the situation, then other genes are recruited directing the elimination of damaged or misfolded protein via proteasome-mediated degradation. Among the latter proteins are the so-called components of the ER associated protein degradation (ERAD) system (Travers et al., 2000, “Functional and genomic analyses reveal an essential coordination between the unfolded protein response and ER-associated degradation, Cell 101(3): 249-58). One of those is ER degradation enhancing a-mannosidase-like protein, which directs the targeting of the misfolded proteins to the proteasome mediated degradation.

If ERSR reaches a certain tipping point, apoptosis is then triggered. Induction of apoptosis during severe ERSR is achieved via at least three different mechanisms. The first is the activation of the transcription of C/EBP homologous protein (CHOP) (for review see Oyadomari et al., 2004). The second is c-Jun NH₂-terminal Kinase (JNK) (Yoneda et al., 2001, “Activation of caspase-12, an endoplastic reticulum (ER) resident caspase, through tumor necrosis factor receptor-associated factor 2-dependent mechanism in response to the ER stress,” J Biol Chem 276(17): 13935-40) activation and the third is activation of ER associated caspase 12 (rat) or 4 (human). These three mechanisms ultimately cause activation of mitochondrial apoptosis signals that converge to activate caspase 3 the final actuator of apoptosis.

An ER stress response or ER stress can be inhibited by modulating the expression or activity of an ER stress response gene or gene product (i.e., a gene or gene product associated with ER stress or an ER stress response, in particular, a gene or gene product that is expressed, produced, up-regulated, or down regulated in response to ER stress). In an embodiment, an ER stress response or ER stress is inhibited by increasing the amount of, or inducing the expression or activity of an ER resident chaperone protein in the cell. In another embodiment, the ER resident chaperone protein is a member of the group stress family, in particular GRP78/BiP. In another embodiment, the ER resident chaperone protein is GRP94, GRP72, Calreticulin, Calnexin, Protein disulfide isomerase, cis/trans-Prolyl isomerase, or HSP47. In another embodiment, an ER stress response is inhibited by inhibiting the expression or activity of, or reducing the amount of a SREBP (e.g. SREBP-1 or SREBP-2) in the cell. In a further embodiment, an ER stress response or ER stress is inhibited by increasing the amount of, or inducing a transcription factor including a Growth Arrest and DNA Damage transcription factor, or a cAMP Response Element Binding (CREB) transcription factor. In a still further embodiment, an ER stress response or ER stress is inhibited by reducing or downregulating the expression or activity of the low density lipoprotein (“LDL”) receptor.

### G. GRP78

Glucose regulated protein (GRP)78 is the main sensor for protein folding and the main actuator of the ERSR. It belongs to heat shock proteins 70 family and is formed by two functional domains. The larger 44 kDa domain possesses ATPase activity. The smaller domain of 20 kDa constitutes the protein binding domain (Chevalier et al., 2002, “Interaction of murine BiP/GRP78 with the DnaJ homologue MTJ1,” J Biol Chem 275(26): 19620-7). A third domain largely composed of helical structure of 10 kDa has unknown functions. GRP78 is constitutively present, although at low levels, in all cultured cells, while expression in vivo is variable depending on the tissues, organs, and developmental age (Li et al., 2006). GRP78, in the absence of unfolded proteins, is in its inactive state bound to ATP. Following binding of unfolded proteins to the 20 kDa moiety, conformational changes trigger ATP hydrolysis in the 44 kDa moiety. The presence of ADP in the 44 kDa subunit increases the affinity for unfolded protein (Chevalier et al., 2000).

In resting conditions GRP78 binds other proteins of which it represents the natural repressor. In particular, it binds to activating transcription factor 6 (ATF6) (Shen et al., 2002), inositol requiring protein (IRE) 1a and PRK-like endoplasmic reticulum kinase (PERK) (Bertolotti et al., 2000, “Dynamic interaction of BiP and ER stress transducers in the unfolded-protein response,” Nat Cell Biol 2(6): 326-32). Once unfolded proteins are sensed, the affinity of GRP78 for the unfolded protein increases and these three proteins are released to be processed for activation or to exert their signaling role. All the three proteins bound and made inactive by GRP78 in non-ERSR conditions participate in the deployment of the ERSR. ERSR in its entirety can be summarized in a four level complex: 1) translation attenuation; 2) transcription of ERSR/UPR specific molecular elements; 3) NF-κB activation; 4) activation of apoptosis.

Activation of GRP78 by binding of unfolded proteins triggers the release of PERK. Free PERK is active and phosphorylates several substrates, such as the eukaryotic inhibitory factor elF2a. Phosphorylation of elF2a de facto inhibits the transcription of a number of cellular proteins. The ultimate goal of this generalized protein transcription synthesis inhibition is to diminish protein input in the ER and therefore ER protein preload, thus, facilitating handling of unfolded protein when this organelle is in stress.

The chaperone activation system is designed to increase protein folding in the ER, when it fails or it is overwhelmed, it targets unfolded proteins toward proteasome-mediated disruption. Accumulation of non functional proteins is potentially a lethal situation. GRP78 up-regulation is seen as a protective mechanism, which prevents activation of programmed cell death by decreasing protein synthesis and increasing protein folding, but also by blocking potentially proapoptotic signals and caspase. However, in certain conditions where GRP78 and other chaperones fail and there is abundance of unfolded proteins, GRP78 fails to repress proapoptotic signals and they are now able to trigger apoptosis. In particular, GRP78 activation and up-regulation has several cellular effects. It has been shown that molecular ablation of GRP78 causes cell death given the critical role of GRP78 in cell repair (Lee et al., 2008, “GRP78 is overexpressed in glioblastomas and regulates glioma cell growth and apoptosis,” Neuro Oncol 10(3): 236-43). Also, overexpression of GRP78 in the absence of ERSR is protective given the role of GRP78 as repressor of proapoptotic factors (Lee et al., 2008). Up regulation of GRP78, during ERSR, reduces unfolded protein presence by working with Ca²⁺ as a chaperone and increasing folding directly. This prevents activation of the apoptotic cascade by the presence of elevated number of unfolded protein. In addition, GRP78 unbound to unfolded proteins directly inhibits proapoptotic components such as Apaf\caspase 9 and prevents procaspase processing and, therefore, their activation (Shiraishi et al., 2006, “ER stress-induced apoptosis and caspase-12 activation occurs downstream of mitochondrial apoptosis involving Apaf-1,” J Cell Sci 119(Pt 19): 3958-66). Secondly, GRP78 binds caspase 12, in murine cells, and can block directly caspase 3 (Reddy et al., 2003, “Endoplasmic reticulum chaperone protein GRP78 protects cells from apoptosis induced by topoisomerase inhibitors: role of ATP binding site in suppression of caspase-7 activation,” J Biol Chem 278(23): 20915-24). Binding to unfolded protein and an increase of GRP78 exerts several additional actions. In fact, the simultaneous release of ATF6, IRE1a and PERK from GRP78 and their subsequent activation, due to competition by unfolded proteins, induces a number of effects, ultimately leading to attenuation of cytotoxicity. In particular, PERK activation via phosphorylation of elF2a turns off most protein synthesis reducing clogging up of protein waiting to be folded in the ER, and activates NF-κB, an anti apoptotic transcription factor. Therefore, a compensated activation of ERSR ultimately prevents cell death, whereas ineffective ERSR compensation triggers programmed cell death.

It is a common view that molecular overexpression of GRP78 is a protective factor (Lee et al., 2008). Also, molecular ablation of GRP78 has been shown to be cytotoxic (Lee et al., 2008). GRP78 expression is induced in several cancer types and is an adaptive change to lack of nutrients and oxygen supply. In fact, in the crowded cancer environment all these conditions are evidently present. The cells live in continuous stress given their own excessive growth. It is believed that GRP78 up regulation represents a significant advantage to prevent death in these conditions. In fact, reports have been presented that show exactly this possibility. GRP78 levels have been found elevated in breast, lung, prostate and liver cancer. Also a correlation has been established between the level of malignancy and the level of GRP78. Several factors can contribute to GRP78 elevation in solid tumors. First of all, glucose decreased availability due to high tissue metabolic rate can signal an increase of GRP78 (glucose regulated protein) (Gatenby 1995, “The potential role of transformation-induced metabolic changes in tumor-host interaction,” Cancer Res 55(18): 4151-6). This effect correlates with the level of vascularization of the neoplasm (Dong et al., 2005, “Vascular targeting and antiangiogenesis agents induce drug resistance effector GRP78 within the tumor microenvironment,” Cancer Res 65(13): 5785-91). In addition, up regulation of GRP78, as seen in several cancers, is associated with direct and indirect inhibition of apoptosis. This is a mechanism that allows cancer cells to survive in their hostile environment. GRP78 activation by unfolded proteins causes its up regulation, and elevated GRP78 levels directly inhibit several steps in the activation of different caspases, the actuators of programmed cell death. In addition, GRP78, through activation of PERK, can activate NF-κB, an anti-apoptotic factor. PERK activation also decreases protein synthesis by inhibiting elF2a, thereby decreasing ER dysfunction (Jiang et al., 2003, “Phosphorylation of the alpha subunit of eukaryotic initiation factor 2 is required for activation of NF-kappaB in response to diverse cellular stresses,” Mol Cell Biol 23(16): 5651-63).

GRP78 elevation has been associated with acquisition of resistance to several chemotherapeutic agents. Typical is the case of the antitumor effect of non steroidal anti-inflammatory drugs and COX2 inhibitors in colorectal cells (Tsutsumi et al., 2004, “Endoplasmic reticulum stress response is involved in nonsteroidal anti-inflammatory drug-induced apoptosis,” Cell Death Differ 11(9): 1009-16). These compound causes cell death by inducing ERSR. In particular, certain COX2 inhibitors, such as celecoxib and its compound dimethylcelecoxib, seem to block SERCA, causing depletion of the ER Ca²⁺ content, misfolding of proteins and apoptosis (Tanaka et al., 2005, “Involvement of intracellular Ca2+ levels in nonsteroidal anti-inflammatory drug-induced apoptosis.” J Biol Chem 280(35): 31059-67). However, in conditions of GRP78 elevation apoptosis is prevented, while reduction of GRP78 potentiates the response to these drugs. Analogously, cisplatin efficacy is amplified by GRP78 inhibition (Zhai et al., 2005, “Decreased cell survival and DNA repair capacity after UVC irradiation in association with down-regulation of GRP78/BiP in human RSa cells.” Exp Cell Res 305(2): 244-52) (Mandic et al., 2003, “Cisplatin induces endoplasmic reticulum stress and nucleus-independent apoptotic signaling,” J Biol Chem 278(11): 9100-6).

Several studies have presented evidence indicating a protective role for GRP78 activation and up-regulation in the brain. In particular, the similarity in terms of regulation between GRP78 and the heat shock protein class is the first hint to this possibility. It has been shown that incapacitation of the GRP78 system leads to an increase of misfolded protein and neurodegeneration (Zhao et al., 2005, “Protein accumulation and neurodegeneration in the woozy mutant mouse is caused by disruption of SILL, a cochaperone of BiP,” Nat Genet. 37(9): 974-9). In addition, GRP78 can protect neurons from oxidative insults by counteracting the effect of Nitric Oxide on depletion of ER Ca²⁺ and consequent apoptosis (Yu et al., 1999, “The endoplasmic reticulum stress-responsive protein GRP78 protects neurons against excitotoxicity and apoptosis: suppression of oxidative stress and stabilization of calcium homeostasis,” Exp Neurol 155(2): 302-14). Another interesting aspect of GRP78 up-regulation in neurons is the fact that it is induced by valproate, an anti seizure agent, whose mechanism of action is unknown, and by lithium a powerful mood stabilizer used in the treatment of bipolar disorders (Shao et al., 2006, “Mood stabilizing drug lithium increases expression of endoplasmic reticulum stress proteins in primary cultured rat cerebral cortical cells.” Life Sci 78(12): 1317-23). The involvement of the GRP78 pathway in the effect of these two drugs in neurons indicates that modulation of this chaperone plays a role in regulating neurophysiologic activities underlying functional brain disorders such as epilepsy and mood disorders. Valproate and other histone deacetylases inhibitors do not increase GRP78 in C6 glioma cells.

Several cell culture studies have produced evidence of cytotoxicity induced by activation of ERSR in glioma cells. Two classes of drugs have been indicated as potentially relevant agents to promote the death of glial tumor cells via activation of ERSR, in the presence of strong up-regulation of GRP78. These molecules includes anti Human immunodeficiency virus drugs of the proteases inhibitor category (Pyrko et al., 2007, “HIV-1 protease inhibitors nelfinavir and atazanavir induce malignant glioma death by triggering endoplasmic reticulum stress.” Cancer Res 67(22): 10920-8) and non steroidal anti-inflammatory drugs (NSIAD) mostly cyclooxygenase type 2 inhibitors and chemically-related compounds. Although they are not chemically related and act through different molecular mechanisms, both of these classes of compounds cause the death of glioma cells in vitro by activating ERSR. Intriguingly, both compound classes cause ERSR, which is signaled by the up regulation of GRP78 expression. It is speculated that HIV protease inhibitors achieve their effect by blocking ubiquitination-signaled, proteasome-mediated misfolded or damaged protein degradation, resulting in excessive non functional, unfolded protein accumulation. This directs ERSR toward apoptosis rather than protection mechanisms, via ultimately caspases activation (Pyrko et al., 2007). In another set of studies, it was shown that ERSR induction by ER Ca²⁺ depletion, caused by COX-2 inhibitors and related molecules devoid of COX-2 inhibitory activity, via the blockade of the smooth endoplasmic reticulum Ca²⁺-ATP-ASE is deadly for glioma cells (Pyrko et al., 2007), and it is again accompanied by GRP78 up-regulation. These data in conjunction with the fact that overexpression of GRP78 in the absence of ERSR induction is cyto-protective, whilst GRP78 molecular ablation with si-RNA causes cell death, indicates that GRP78 is not directly toxic in these cells, but rather the environment in which ERSR take place determines the sensibility toward toxicity (Lee et al., 2008). This data and the finding that Ca²⁺ handling is deregulated in glioma cells as compared to their primary counterpart prompted the analysis of the differences in ERSR in normal astrocytes and glioma cells, such as C6 and U87-MG looking at GRP78 expression, profile of response and gliotoxicity. This analysis shows that there is a clear difference in Ca²⁺ handling between astrocytes and glioma cells, such as C6. These differences can be reflected in abnormalities of ERSR in rat glioma cells as compared to their normal counterpart the astrocytes. These differences can trigger enhanced apoptotic cell death in glioma cells when ERSR is induced rather than a protective effect. This study's observations were extended to human glioma cells. In particular, glioma cells are particularly sensitive to ERSR and up regulate GRP78 at a higher level than astrocytes. Although, this has been shown to be the base for adaptation and survival to a number of insults, in glioma cells this reflects in increased sensitivity to cell death during ERSR and, therefore, makes GRP78 an attractive biosensor to reveal potential activators of ERSR. Data from this study indicats that compounds identified via such an approach are relevant gliotoxic agents, further indicating a relative failure to cope with ERSR glioma cells.

Further, during ERSR there are several events that converge to cause the activation of NF-κB, a transcription factor involved in several cellular phenomenon that also antiapoptotic properties. If ERSR is caused by release of Ca²⁺ from the ER to the cytoplasm, activation of Ca²⁺ sensitive, or Ca²⁺-diacylglycerol sensitive protein kinase C isoforms, via the phosphorylation of the inhibitor of NF-κB (IKB) (Pahl et al., 1996, “Activation of NF-kappa B by ER stress requires both Ca2+ and reactive oxygen intermediates as messengers.” FEBS Lett 392(2): 129-36), causes NF-κB activation and its nuclear translocation to activate transcription of target genes. Other pathways downstream to GRP78 activation can concur to further activate NF-κB. For example phosphorylation via PERK of elF2a via a not known mechanism causes the activation of NF-κB (Jiang et al., 2003). Thus, disclosed herein are methods of inhibiting ER stress and ERSR while increasing ER chaperone proteins like (GRP)78 to treat gliomas.

Glial cells comprise a large proportion of the total cell population in the CNS. Unlike neurons, glial cells retain the ability to proliferate postnatally, and some glial cells still proliferate in the adult or aged brain. Uncontrolled glial proliferation can lead to aggressive primary intracranial tumors, the vast majority of which are astrocytomas, and therefore, of glial origin. Tumors of astrocytic origin vary widely in morphology and behavior, and, according to the 1993 World Health Organization (WHO) classification schemae, can be separated into three subsets. Astrocytomas, the lowest grade tumors, are generally well-differentiated and tend to grow slowly. Anaplastic astrocytomas are characterized by increased cellularity, nuclear pleomorphism, and increased mitotic activity. They are intermediate grade tumors and show a tendency to progress to a more aggressive grade. Glioblastomas are considered the most aggressive, with poorly differentiated cells, vascular proliferation, and necrosis.

### H. METHODS

In various aspects, the invention is related to administration of acridine analogs for, inter alia, the treatment of gliomas, for inhibiting intracranial metastasis of gliomal cancer cells, and/or for prevention of relapse of glioma in a subject.

1. Treatment of Gliomas

Disclosed herein are methods of treating a subject with a glioma, comprising administering to the subject an acridine analog or a pharmaceutically acceptable salt or hydrate thereof, wherein the acridine analog has formula IA or IB:

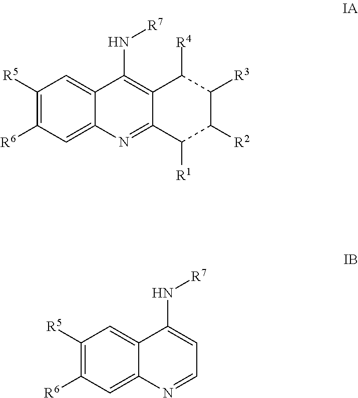

wherein the dashed line is either a single or double bond; R¹, R², R³, and R⁴ are, independently of one another, hydrogen, amino, halide, hydroxy, optionally substituted alkyl, or optionally substituted alkoxy; R⁵ represents hydrogen, amino, halide, hydroxy, methoxy, or ethoxy; R⁶ represents hydrogen, halide, hydroxy, methoxy, or ethoxy; and R⁷ represents a hydrogen, optionally substituted alkyl, or aminoalkyl, or a pharmaceutically acceptable salt or hydrate thereof, wherein R⁷ is —CH(CH₃)(CH₂)₃NEt₂ or —CH(CH₃)(CH₂)₃N(Et)(EtOH).

In one aspect, the acridine analog comprises 9-aminoacridine, 4-aminoquinoline, chloroquine, hydroxychloroquine, 3-chloro-N⁹-(5-(diethlyamino)pentan-2-yl)-7-methoxy acridine-2,9-diamine, or amodiaquine. In a further aspect, the acridine analog comprises 9-aminoacridine. In a further aspect, the acridine analog comprises quinacrine.

Additionally, the acridine analog or a pharmaceutically acceptable salt or hydrate thereof can be administered at a dosage of 1 to about 500 mg/kg of the subject. In one aspect, the acridine analog or a pharmaceutically acceptable salt or hydrate thereof can be administered at a dosage of 10 to about 200 mg/kg of the subject. In a further aspect, the acridine analog or a pharmaceutically acceptable salt or hydrate thereof can be administered at a dosage of 10 to about 100 mg/kg of the subject. In a yet further aspect, the acridine analog or a pharmaceutically acceptable salt or hydrate thereof can be administered at a dosage of 20 to about 500 mg/kg of the subject.

Additionally, disclosed herein is a method of treating a subject with a glioma, comprising administering to the subject a GRP protein or variant thereof.

2. Inhibiting Intracranial Metastasis of Gliomal Cancer Cells

Also disclosed herein, is a method of inhibiting intracranial metastasis of gliomal cancer cells in a subject, comprising administering to the subject an acridine analog or a pharmaceutically acceptable salt or hydrate thereof, wherein the acridine analog has formula IA or IB:

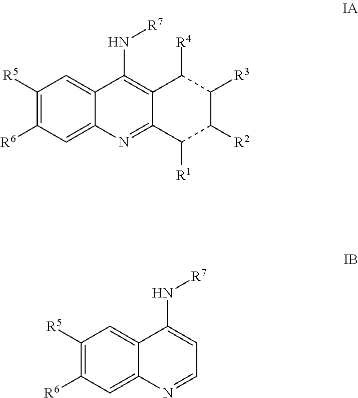

wherein the dashed line is either a single or double bond; R¹, R², R³, and R⁴ are, independently of one another, hydrogen, amino, halide, hydroxy, optionally substituted alkyl, or optionally substituted alkoxy; R⁵ represents hydrogen, halide, amino, hydroxy, methoxy, or ethoxy; R⁶ represents hydrogen, halide, hydroxy, methoxy, or ethoxy; and R⁷ represents a hydrogen, optionally substituted alkyl, or aminoalkyl, or a pharmaceutically acceptable salt or hydrate thereof, wherein R⁷ is —CH(CH₃)(CH₂)₃NEt₂ or —CH(CH₃)(CH₂)₃N(Et)(EtOH).

In one aspect, the acridine analog comprises 9-aminoacridine, 4-aminoquinoline, chloroquine, hydroxychloroquine, 3-chloro-N⁹-(5-(diethlyamino)pentan-2-yl)-7-methoxy acridine-2,9-diamine, or amodiaquine. In a further aspect, the acridine analog comprises 9-aminoacridine. In a further aspect, the acridine analog comprises quinacrine.

Additionally, the acridine analog or a pharmaceutically acceptable salt or hydrate thereof can be administered at a dosage of from about 0.1 to 500 mg/kg of the subject. In one aspect, the acridine analog or a pharmaceutically acceptable salt or hydrate thereof can be administered at a dosage of 0.1 to about 200 mg/kg of the subject. In a further aspect, the acridine analog or a pharmaceutically acceptable salt or hydrate thereof can be administered at a dosage of 0.1 to about 100 mg/kg of the subject. In yet a further aspect, the acridine analog or a pharmaceutically acceptable salt or hydrate thereof can be administered at a dosage of 0.1 to about 500 mg/kg of the subject.

Furthermore, disclosed herein is a method of treating a glioma in a subject, comprising administering to the subject a composition that increases ER stress response or ER stress and the expression or activity of an ER resident chaperone protein.

In one aspect, the ER resident chaperone protein is a member of the group stress family GRP78/BiP. In a further aspect, the ER resident chaperone protein is GRP94, GRP72, Calreticulin, Calnexin, protein disulfide isomerase, cis/trans-prolyl isomerase, or HSP47.

Also disclosed herein is a method of treating a glioma in a subject, comprising administering to the subject a composition that increases the expression or activity of a SREBP and causes an ER stress response or ER stress, wherein the SREBP is SREBP-1 or SREBP-2.

Additionally, disclosed here in is a method of treating a glioma in a subject, comprising administering to the subject a composition that induces Growth Arrest and DNA Damage transcription factor or a cAMP Response Element Binding (CREB) transcription factor and causes an ER stress response or ER stress.

Moreover, disclosed herein is a method of treating a glioma in a subject, comprising administering to the subject a composition that upregulates the expression or activity of the low density lipoprotein receptor and causes an ER stress response or ER stress.

Also disclosed herein is a method of inhibiting the growth of a glioma cell in vitro, comprising contacting the glioma cell with a composition that causes ER stress response or ER stress.

In one aspect, the glioma cell is from the C6, U251, or U87-MG cell line. In a further aspect, the glioma cell is contacted with a composition that increases the expression or activity of an ER resident chaperone protein and inhibits an ER stress response or ER stress. In a further aspect, the glioma cell is contacted with a composition that inhibits the expression or activity of a SREBP and inhibits an ER stress response or ER stress. In yet a further aspect, the glioma cell is contacted with a composition that induces Growth Arrest and DNA Damage transcription factor or a cAMP Response Element Binding (CREB) transcription factor and causes an ER stress response or ER stress. Additionally, the glioma cell is contacted with a composition that downregulates the expression or activity of the low density lipoprotein receptor and causes an ER stress response or ER stress.

Furthermore, disclosed herein is a method of screening a compound for putative activity against glioma, comprising: (a) contacting a cell with a candidate compound; (b) assaying a level of ER stress or ER stress response of the cell in the presence of the candidate compound; wherein an increase in the level of ER stress or ER stress response, as compared to a control, indicates a compound having putative activity against glioma.

In one aspect, assaying a level of ER stress or ER stress response comprises measuring the level of expression or activity of an ER resident chaperone protein, wherein the ER resident chaperone protein is a member of the group stress family GRP78/BiP. In a further aspect the ER resident chaperone protein is GRP94, GRP72, Calreticulin, Calnexin, protein disulfide isomerase, cis/trans-prolyl isomerase, or HSP47. Additionally, assaying a level of ER stress or ER stress response comprises measuring expression or activity of a SREBP, wherein the SREBP is SREBP-1 or SREBP-2. Assaying a level of ER stress or ER stress response also comprises measuring an amount of Growth Arrest and DNA Damage transcription factor or a cAMP Response Element Binding (CREB) transcription factor. Furthermore, assaying a level of ER stress or ER stress response comprises measuring expression or activity of the low density lipoprotein receptor.

3. Prevention of Relapse of Glioma in a Subject

In one aspect, the invention relates to a method of preventing relapse in a subject previously treated for a glioma, the method comprising administering to the subject a prophylactically effective amount of an acridine analog or a pharmaceutically acceptable salt or hydrate thereof. The acridine analog can have a structure of formula IA or IB:

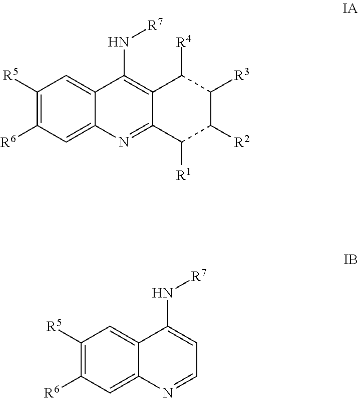

wherein the dashed line is either a single or double bond; R¹, R², R³, and R⁴ are, independently of one another, hydrogen, amino, halide, hydroxy, optionally substituted alkyl, or optionally substituted alkoxy; R⁵ represents hydrogen, amino, halide, hydroxy, methoxy, or ethoxy; R⁶ represents hydrogen, halide, hydroxy, methoxy, or ethoxy; and R⁷ represents a hydrogen, optionally substituted alkyl, or aminoalkyl, or a pharmaceutically acceptable salt or hydrate thereof. In a further aspect, R⁷ is —CH(CH₃)(CH₂)₃NEt₂ or —CH(CH₃)(CH₂)₃N(Et)(EtOH). In a further aspect, the acridine analog comprises 9-aminoacridine, 4-aminoquinoline, chloroquine, hydroxychloroquine, 3-chloro-N⁹-(5-(diethlyamino)pentan-2-yl)-7-methoxy acridine-2,9-diamine, or amodiaquine. In a further aspect, the acridine analog comprises 9-aminoacridine. In a further aspect, the acridine analog comprises quinacrine.

### I. COMPOSITIONS

The compositions that can be used herein are those that causes ER stress response or ER stress by, e.g. modulating GRP78 expression and/or activity in a cell. Numerous agents for modulating expression/activity of intracellular proteins such as GRP in a cell are known. Typical agents for promoting (e.g., agonistic) activity of GRPs include mutant/variant GRP polypeptides or fragments, nucleic acids encoding a functional GRP polypeptide or variant, and small organic or inorganic molecules.

1. Acridine Analogs Thereof

In particular examples, molecules that modulate ERSR and ER stress are aminoacridine analogs, including quinaline analogs, and pharmaceutically acceptable salts thereof, including, 9-aminoacridine and analogs like quinacrine, as well as 4-aminoquinoline and analogs such as chloroquine, hydroxychloroquine, and amodiaquine. The disclosed compounds can be used in methods for inhibiting glioma growth in vitro, or growth and matastisis in vivo. Generally, the methods comprise administering an effective amount of an aminoacridine compound or pharmaceutically acceptable salt thereof in an amount effective to inhibit glioma growth and/or metastasis. Further examples of suitable aminoacridine analogs are shown in Formulas IA or IB:

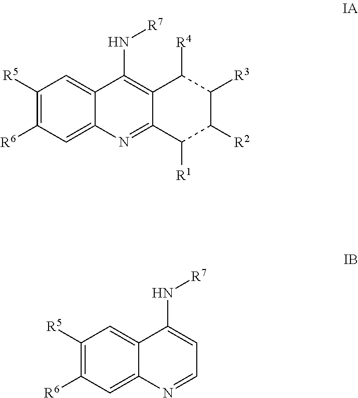

wherein the dashed line is either a single or double bond; R¹, R², R³, and R⁴ are, independently of one another, hydrogen, amino, halide, hydroxy, optionally substituted alkyl, or optionally substituted alkoxy; R⁵ represents hydrogen, aminohalide, hydroxy, methoxy, or ethoxy; R⁶ represents hydrogen, halide, hydroxy, methoxy, or ethoxy; and R⁷ represents a hydrogen, optionally substituted alkyl, or aminoalkyl, or a pharmaceutically acceptable salt or hydrate thereof. In certain examples R⁷ can be —CH(CH₃)(CH₂)₃NEt₂ or —CH(CH₃)(CH₂)₃N(Et)(EtOH). Each of the foregoing alkyl groups can be straight chain or branched.

A specific example of an aminoacridine includes the 9-aminoacridine Mepacrine, which is otherwise known as quinacrine. Also, disclosed are pharmaceutically acceptable salts of quinacrine, as well as analogs, 9-aminoacridine hydrochloride hydrate and OSSL 053454 (3-chloro-N⁹-(5-(diethlyamino)pentan-2-yl)-7-methoxy acridine-2,9-diamine). Quinacrine hydrochloride is described by the following formulas: N⁶-(6-chloro-2-methoxy-9-acridinyl)-N¹, N¹-diethyl-1,4-pentanediamine dihydrochloride; 6-chloro-9-((4-(diethylamino)-1-methylbutyl)amino)-2-methoxyacridine dihydrochloride; and 3-chloro-7-methoxy-9-(1-methyl-4-diethyl aminobutylamino) acridine dihydrochloride. It is available in formulations for pharmaceutical use under a number of trade designations, including ATABRINE™ Hydrochloride (Sanofi Winthrop Pharmaceuticals, New York, N.Y.).

Clinically, quinacrine hydrochloride is employed for the treatment of giardiasis and cestodiasis. In addition, it has been used for the treatment and suppression of malaria. Quinacrine hydrochloride has been reported to be a phospholipase A₂ (PLA₂) inhibitor in platelets (Beckman and Seferynska, “Possible involvement of phospholipase activation in erythroid progenitor cell proliferation,” Exp Hematol 17:309, 1989). Coinjection of quinacrine hydrochloride with PLA₂ reduced the inflammogenic potency of the latter by 64%, indicating that quinacrine hydrochloride might have some anti-inflammatory activity (Moreno et al., 1992, “PLA₂-induced oedema in rat skin and histamine release in rat mast cells. Evidence for involvement of lysophospholipids in the mechanism of action,” Agents Actions 36:258). In addition, quinacrine has been found to prevent the alpha-granule release reaction of platelets (Prowse et al., 1982, “Prevention of the platelpha-granule release reaction by membrane-active drugs,” Thrombosis Research 25:219).

In clinical practice, quinacrine has been used for pleurodesis through intrapleural administration. When the material was administered at 100 mg/day in 50 ml saline for 4 days, the peak concentration reported was less than 10 ng/ml (Janzing et al., 1993, “Intrapleural quinacrine instillation for recurrent pneumothorax or persistent air leak,” Ann Thoracic Surgery 55:368). The intraperitoneal toxicity of quinacrine in rats has also been examined (Senoir et al., 1984, “Morphometric and kinetic studies on the change induced in the intestinal mucosa of rats by intraperitoneal administration of quinacrine,” Cell & Tissue Kinetics 17:445, 1984); in these animals. 12 mg (equivalent to 48 mg/kg) was given to rats each day for 4 days, for a total of 192 mg/kg.

9-aminoacridine and pharmaceutically acceptable salts thereof has been used as therapeutic agent since 1942. Certain 9-aminoacridine compounds have been believed to be intercalating capable of DNA damaging activity; however, 9-aminoacridine and quinacrine were not found to show DNA damaging activity (US Patent Application No. 2007/0270455). Both 9-aminoacridine and quinacrine were found to be more toxic to tumor than to normal cells in vitro and in vivo. Moreover, both compounds were shown to be capable of p53 activation and p53-dependent killing of a variety of tumor cell types, besides RCC. p53 dependence of their anti-tumor activity clearly distinguishes the aminoacridines from conventional chemotherapeutic drugs based on their targeting of tumors with wild type or functional p53.

Aminoacridines do not fit any known category of p53 activating agents. Although they may cause accumulation of p53, they do not induce p53 phosphorylation, unlike DNA damaging drugs. Moreover, aminoacridines do not cause DNA damage. Instead, the primary effect of aminoacridines appeared to be not p53 activation but repression of NF-κB, which later leads to p53 induction. Importantly, inhibition of NF-κB activates p53 function in a cell in which it cannot be “waked up” by any of the direct approaches to p53 activation, including introduction of Arf, knockdown of Hdm2 or ectopic overexpression of p53.

Inhibition of NF-κB is usually achieved through stabilization of the main negative regulator of NF-κB, IκB. Genetically, it can be done by mutating regulatory phosphorylation sites of this protein and pharmacologically—through inhibition of upstream kinases leading to a block of IκB phosphorylation. Many known chemical inhibitors of NF-κB act through this mechanism. Stabilization of IκB results in cytoplasmic sequestration and functional inactivation of NF-κB complexes as transcription factors.

The activity of aminoacridines can be superior to previous drugs since they promote strong accumulation of NF-κB complexes in the nuclei in response to activating stimuli accompanied with a complete repression of transactivation. Hence, aminoacridines and analogs thereof can inhibit NF-κB by acting downstream of IκB and involving conversion of NF-κB into an inactive complex. The lack of NF-κB-dependent transcription can lead to the depletion of the pool of IκB (that is a direct transcription target of NF-κB) and retention of NF-κB in the nucleus due to the lack of nuclear export, normally exerted by IκB. Interestingly, the knockout of any of the cellular factors involved in NF-κB activation (IKKα, IKKβ, TBK1, and PKC-zeta) does not imitate the effect of aminoacridines, indicating that none of them is a target of aminoacridines or analogs thereof. It has recently been demonstrated that nuclear accumulation of inactive NF-κB complexes, containing p65, occurs after cell treatment with UV, doxorubicin and daunorobicin; however, none of these treatments is comparable with aminoacridines in activating p53, presumably due to weaker NF-κB inhibitory activity.

The aminoacridines can be effective not only against the IκB phosphorylation arm of NF-κB signaling (“canonical” NF-κB activation pathway), but also through alternative mechanisms of NF-κB activation. This is supported by the ability of aminoacridines, such as 9-aminoacridine, to block stimulated NF-κB activity and also effectively reduce basal levels of constitutive NF-κB activity in tumor cells. By contrast, IKK2 inhibitors are only able to block stimulated NF-κB activity.

2. Peptides

It is contemplated herein that GRP78 activity following ER stress induction can be modulated by other proteins including other native GRP proteins as well as GRP protein variants. For example, other native GRP proteins can upregulate GRP78 activity and GRP protein variants can compete with a native GRP protein for binding ligands such as a caspase (e.g., to downregulate apoptosis).

It is further contemplated herein that GRP protein variants can be generated through various techniques known in the art. For example, GRP78 protein variants can be made by mutagenesis, such as by introducing discrete point mutation(s), or by truncation (e.g., of the transmembrane region). Such mutations can give rise to a GRP78 variant or fragment having substantially the same, improved, or merely a subset of the functional activity of a native GRP78 protein. Agonistic (or superagonistic) forms of the protein may be generated that constitutively conduct one or more GRP78 functional activities. Other variants of GRP polypeptides that can be generated include those that are resistant to proteolytic cleavage, as for example, due to mutations which alter protease target sequences. The determination of whether a change in the amino acid sequence of a peptide results in a GRP78 protein variant having one or more of the functional activities of a native GRP78 protein can be readily determined by testing the variant for any one or more of the native GRP78 protein functional activities, such as modulating apoptosis by decreasing protein synthesis, increasing protein folding, or binding caspase.

As discussed herein there are variants of the GRP78 protein that are known and herein contemplated. Protein variants and compounds are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein compounds, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| Alanine | Ala, A |
| Allosoleucine | AIle |
| Arginine | Arg, R |
| Asparagines | Asn, N |
| Aspartic acid | Asp, D |
| Cysteine | Cys, C |
| Glutamic acid | Glu, E |
| Glutamine | Gln, K |
| Glycine | Gly, G |
| Histidine | His, H |
| Isolelucine | Ile, I |
| Leucine | Leu, L |
| Lysine | Lys, K |
| Phenylalanine | Phe, F |
| Proline | Pro, P |
| Serine | Ser, S |
| Threonine | Thr, T |
| Tyrosine | Tyr, Y |
| Tryptophan | Trp, W |
| Valine | Val, V |

TABLE 2
Amino Acid Substitutions

| | Exemplary Conservative Substitutions, |
|---|---|
| Original Residue | others are known in the art |
| Ala | ser |
| Arg | lys, gln |
| Asn | gln; his |
| Asp | glu |
| Cys | ser |
| Gln | asn, lys |
| Glu | asp |
| Gly | ala |
| His | asn; gln |
| Ile | leu; val |
| Leu | ile; val |
| Lys | arg; gln; |
| Met | leu; ile |
| Phe | met; leu; tyr |
| Ser | thr |
| Thr | ser |
| Trp | tyr |
| Tyr | trp; phe |
| Va | lile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g., Arg, are accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 (1983)), acetylation of the N-terminal amine and, in some instances, amidation of the C-terminal carboxyl.

### J. USING THE COMPOSITIONS

The disclosed acridine analogs or can be given to a subject with glioma. Any subject in need of the quinacrine and acridine analogs as disclosed herein can be given the aminoacridines. The subject can, for example, be a mammal, such as a mouse, rat, rabbit, hamster, dog, cat, pig, cow, sheep, goat, horse, or primate, such as monkey, gorilla, orangutan, chimpanzee, or human.

The disclosed compositions can be used for inhibiting glioma cell proliferation and cause glioma cell death. Inhibiting glioma cell proliferation means reducing or preventing glioma cell growth. Inhibitors can be determined by using a cancer cell assay. For example, the C6, U251 or U87-MG cell line can be cultured on 96-well plates in the presence or absence of aminoacridines for 48 hours. The cells can be fixed, stained with crystal violet, solubilized in deoxycholate, and read in a spectrophotometer at 590 nm. In certain examples, the compositions are those that will inhibit 10%, 15%, 20%, 25%, 30%, 35%, 40%, 45%, 50%, 55%, 60%, 65%, 70%, 75%, 80%, 85%, 90%, or 95% of the cell growth relative to a control as determined by spectrophotometry, where any of the stated values can form an upper or lower endpoint of a range.

As disclosed herein, acridine analogs or can be used for promoting glioma cell apoptosis. Promoting glioma cell apoptosis means causing the cell to die. An apoptosis assay can be used to determine if quinacrine and acridine analogs promote glioma cell apoptosis. The percent of apoptosis can be determined as the percent of annexin V-positive, propidium iodide-negative cells of the total cells counted in an apoptosis assay. The disclosed compositions can cause at least about 10%, 15%, 20%, 25%, 30%, 35%, 40%, 45%, 50%, 55%, 60%, 65%, 70%, 75%, 80%, 85%, 90%, or 95% of the cells to be apoptotic, where any of the stated values can form an upper or lower endpoint of a range.

The acridine analogs disclosed herein can also be used for inhibiting metastasis of cancer cells. Inhibiting metastasis of cancer cells means decreasing or lowering the amount of metastatic tumors that arise in an organism. For example, disclosed are compositions that inhibit metastasis in an in vivo assay. One way of performing an in vivo assay to determine if an inhibitor inhibits metastasis is to inject a cancer cell line, such as U87-MG, into the intracranial cavity of a mouse. Mice are pretreated with the inhibitor or a control intraperotneally, for example. The mouse can then be treated regularly, for example, daily with vehicle for a period of time, for example, 21 days. The mouse can then be sacrificed and assayed for metastatic tumor formation. Disclosed are compositions which inhibit metastatic tumor formation in this type of assay disclosed herein, as well as compositions that reduce metastatic tumor formation by at least about 10%, 15%, 20%, 25%, 30%, 35%, 40%, 45%, 50%, 55%, 60%, 65%, 70%, 75%, 80%, 85%, 90%, or 95% relative to a control compound, where any of the stated values can form an upper or lower endpoint of a range. For example, the disclosed compounds can inhibit intracranial metastasis arising from, for example, gliomal cancer.

The disclosed acridine analogs can be used for inhibiting tumorigenesis. Inhibiting tumorigenesis means decreasing or lowering the amount of tumors present in an organism. For example, disclosed are compositions that inhibit tumorigenesis in an in vivo assay. One way of performing an in vivo assay to determine if an inhibitor inhibits tumorigenesis is to inject a cancer cell line subcutaneously, such as U87-MG, into a mouse. The mouse can then be treated regularly, for example, twice weekly with vehicle or quinacrine and acridine analogs for a period of time, for example, 21 days or 28 days. The mouse can then be sacrificed and assayed for tumor formation and size. Disclosed are compositions that inhibit tumorigenesis in this type of assay disclosed herein, as well as compositions that reduce tumorigenesis by at least about 10%, 15%, 20%, 25%, 30%, 35%, 40%, 45%, 50%, 55%, 60%, 65%, 70%, 75%, 80%, 85%, 90%, or 95% relative to a control compound, where any of the stated values can form an upper or lower endpoint of a range.

The disclosed acridine analogs and (GRP)78 and its variants can also be administered to cells to induce the apoptosis of the cells by activating the GPR78/ERSR pathway. ERSR activation in glioma cells triggers cell death and is accompanied by increasing GPR78 elevation. Quinacrine can increase expression of GRP78 gene expression while being gliotoxic in rat and human glioma cell lines. The disclosed compositions can promote apoptosis of glioma cells at concentrations of at least about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 μM, where any of the stated values can form the upper or lower endpoint of a range.

When the compositions are administered, they typically cause the appearance of active fragments of proapoptotic proteins. Generation of pro-apoptotic proteins can be determined by any means for determining their presence. For example, standard biotechnology methods such as PCR or Northern blots can be used to determine the expression levels of pro-apoptotic genes. A decrease can be determined by assaying the expression levels of a desired anti-apoptotic gene in the presence of a potential inhibitor and comparing this level of expression to the level of expression in the absence of the inhibitor. Disclosed are inhibitors, which increase the expression of the pro-apoptotic genes in such an assay.

As discussed herein, acridine analogs can, for example, be used to reduce the proliferation of glioma cells, as well as to cause the apoptosis of glioma cells or inhibit the readhesion of glioma cells or inhibit brain spreading of glioma cells. Quinacrine, for example, can be administered to any cancer cell that responds to depletion of Ca²⁺ stores and protein unfolding by activating GRP78/ERSR.

It is understood that certain cancers can give rise to cancer cell lines. Typically, cancer cell lines are cells that are maintained in cell culture, but that arose from a specific type of cancer. Quinacrine and acridine analogs can be used for a variety of cancers, but can, for example, be used for cancers that are related to the U87-MG cancer cell line and the C6 cancer cell line. The U87-MG cancer cell line and the C6 cancer cell line arose from glioma cells. Also disclosed are cancer cell lines having the properties of the U87-MG cancer cell line and the C6 cancer cell line.

The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, stomach cancer, head and neck carcinoma, large bowel cancer, hematopoietic cancers; testicular cancer; colon and rectal cancers, prostatic cancer, or pancreatic cancer.

Compounds disclosed herein can also be used for the treatment of precancer conditions such as cervical and anal dysplasias, other dysplasias, severe dysplasias, hyperplasias, atypical hyperplasias, and neoplasias.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. In a preferred aspect, the compositions disclosed herein can be administered intraperitoneally. For example, during intraabdominal surgery (e.g., to resection of a tumor), the disclosed compositions can be used as a wash. In this way, cancer cells that were not excised and/or cells that may be unattached but still present in the body, can be killed upon readhereing. Alternatively, the disclosed compositions can be administered around the time of surgery (peri-operative), before surgery (pre-operative), or after surger (post-operatively). The compounds can be administered a week, 2-5 days, 1-3 days, 1-18 hours, 1-12 hours, 1-6 hours, or less than an hour before or after tumor resection surgery.

In another preferred example, the disclosed compositions can be administered by I.V., by injection and/or an I.V. drip.

The disclosed compositions can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem 2:447-451, 1991; Bagshawe, Br J Cancer, 60:275-281, 1989; Bagshawe et al., Br J Cancer 58:700-703, 1988; Senter et al., Bioconjugate Chem 4:3-9, 1993; Battelli et al., Cancer Immunol Immunother 35:421-425, 1992; Pietersz and McKenzie, Immunolog Reviews 129:57-80, 1992; and Roffler et al., Biochem Pharmacol, 42:2062-2065, 1991). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research 49:6214-6220, 1989; and Litzinger and Huang, Biochimica et Biophysica Acta, 1104:179-187, 1992). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

2. Pharmaceutically Acceptable Carriers

The compositions can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

3. Dosage

For therapeutic uses, pharmaceutical compositions and formulations can contain an effective amount of active for treating the disorder. The specific effective amount for any particular subject will depend upon a variety of factors including the disorder being treated and the severity of the disorder; the identity and activity of the specific composition employed; the age, body weight, general health, sex and diet of the patient; the time of administration; the route of administration; the rate of excretion of the specific composition employed; the duration of the treatment; drugs used in combination or coincidental with the specific composition employed and like factors well known in the medical arts. For example, it is well within the skill of the art to start doses of a composition at levels lower than those required to achieve the desired therapeutic effect and to gradually increase the dosage until the desired effect is achieved. One can also evaluate the particular aspects of the medical history, signs, symptoms, and objective laboratory tests that are known to be useful in evaluating the status of a subject in need of attention for the treatment of ischemia-reperfusion injury, trauma, drug/toxicant induced injury, neurodegenerative disease, cancer, or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: 1) a subject's physical condition is shown to be improved, 2) the progression of the disease or condition is shown to be stabilized, or slowed, or reversed, or 3) the need for other medications for treating the disease or condition is lessened or obviated, then a particular treatment regimen will be considered efficacious. If desired, the effective daily dose can be divided into multiple doses for purposes of administration. Consequently, single dose compositions can contain such amounts or submultiples thereof to make up the daily dose.

An effective amount of the composition can also be determined by preparing a series of compositions comprising varying amounts of the peptide-lipid conjugates and determining the release characteristics in vivo and in vitro and matching these characteristics with specific pharmaceutical delivery needs, inter alia, subject body weight, disease condition and the like.

The dosage for the compositions can be adjusted by the individual physician or the subject in the event of any counterindications. Dosage can vary, and can be administered in one or more dose administrations daily, for one or several days. Guidance can be found in the literature for appropriate dosages for given classes of pharmaceutical products.

The dosage ranges for the administration of the compositions are those large enough to produce the desired effect in which the symptoms disorder is affected. The dosage should not be so large as to cause adverse side effects, such as unwanted cross-reactions, anaphylactic reactions, and the like. Generally, the dosage can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 mg/kg, where any of the stated values can be upper of lower end points of a range.

Example dosages are disclosed herein. For example, when treating a subject with a glioma, when inhibiting intracranial metastasis of gliomal cancer cells in a subject, or when preventing relapse of glioma in a subject, the acridine analog or a pharmaceutically acceptable salt or hydrate thereof can be administered at a dosage of from 0.1 to about 500 mg/kg of the subject, can be administered at a dosage of from 0.1 to about 200 mg/kg of the subject, can be administered at a dosage of from 0.1 to about 100 mg/kg of the subject, or can be administered at a dosage of from 0.1 to about 500 mg/kg of the subject.

In other examples, the disclosed compositions can be encapsulated in a microparticle in order to control the release of the composition.

4. Single-Agent Therapy

In various aspects, the disclosed acridine analogs can be adminstered to subjects. For example, treating a subject with a glioma can be effected by a method comprising administering to the subject an acridine analog or a pharmaceutically acceptable salt or hydrate thereof. For example, inhibiting intracranial metastasis of gliomal cancer cells in a subject can be effected by a method comprising administering to the subject an acridine analog or a pharmaceutically acceptable salt or hydrate thereof. For example, preventing relapse of glioma in a subject can be effected by a method comprising administering to the subject an acridine analog or a pharmaceutically acceptable salt or hydrate thereof.

5. Combination Therapy

The compositions can be administered alone or combination with chemotherapeutic drugs. Combination therapy can present advantages over single-agent therapies: lower treatment failure rate, lower case-fatality ratios, slower development of resistance and consequently, less money needed for the development of new drugs. Chemotherapeutic drugs include conventional chemotherapeutic reagents such as alkylating agents, anti-metabolites, anti-mitototics, plant alkaloids, antibiotics, and miscellaneous compounds. Examples of these drugs include CDDP, methotrexate, vincristine, adriamycin, bleomycin, carmustine, hydroxyurea, hydrazine, nitrosoureas, triazenes such as dacarabzine and temozolomide, nitrogen mustards such as chlorambucil, cyclophosphamide, isofamide, mechlorethamine, melphalan, uracil mustard; aziridine such as thiotepa; methanesulphonate esters such as busulfan; platinum complexes such as cisplatin, carboplatin; bioreductive alkylators, such as mitomycin and altretemine. Chemotherapeutic drugs also include proteasome inhibitors such as salinosporamides, bortezomib, PS-519, and omuralide. The disclosed compounds can also be administered in combination with surgery. For example, the disclosed compounds can be administered prior to, during or after surgery or radiotherapy. Administration during surgery can be as a bathing solution for the operation site. The resected tumor can also be bathed in the disclosed compounds.

Thus in various further aspects, the disclosed acridine analogs can be adminstered to subjects in combination with one or more chemotherapeutic drugs. For example, treating a subject with a glioma can be effected by a method comprising administering to the subject an acridine analog or a pharmaceutically acceptable salt or hydrate thereof in combination with one or more chemotherapeutic drugs. For example, inhibiting intracranial metastasis of gliomal cancer cells in a subject can be effected by a method comprising administering to the subject an acridine analog or a pharmaceutically acceptable salt or hydrate thereof in combination with one or more chemotherapeutic drugs. For example, preventing relapse of glioma in a subject can be effected by a method comprising administering to the subject an acridine analog or a pharmaceutically acceptable salt or hydrate thereof in combination with one or more chemotherapeutic drugs.

It is contemplated that acridine analogs can be adminstered before, simultaneously, or after the administration of one or more chemotherapeutic drugs. While not wishing to be bound by theory, it is believed that acridine analogs, in combination with one or more chemotherapeutic drugs, can have an augmented or synergystic effect on the subject. Further, acridine analogs, in combination with one or more chemotherapeutic drugs, can be individually given in dosages lower than the one or more chemotherapeutic drugs would be typically adminstered as single-agent therapies.

In further aspects, the invention relates to administration of the disclosed acridine analogs to subjects in combination with Temozolomide (4-methyl-5-oxo-2,3,4,6,8-pentazabicyclo [4.3.0] nona-2,7,9-triene-9-carboxamide). For example, treating a subject with a glioma can be effected by a method comprising administering to the subject an acridine analog or a pharmaceutically acceptable salt or hydrate thereof in combination with Temozolomide. For example, inhibiting intracranial metastasis of gliomal cancer cells in a subject can be effected by a method comprising administering to the subject an acridine analog or a pharmaceutically acceptable salt or hydrate thereof in combination with Temozolomide. For example, preventing relapse of glioma in a subject can be effected by a method comprising administering to the subject an acridine analog or a pharmaceutically acceptable salt or hydrate thereof in combination with Temozolomide.

It is also understood that the disclosed acridine analogs, when adminstered to subjects in combination with one or more chemotherapeutic drugs, can also be employed in connection with radiation therapy and/or surgical therapy.

6. Use in Connection with Radiotherapy

Radiation therapy (Radiotherapy), including Brachytherapy, can be used to treat gliomas. In one aspect, the invention relates to the administration of the disclosed acridine analogs to subjects in connection with radiation therapy. It is contemplated that acridine analogs can be adminstered before, during, or after the radiation therapy. For example, treating a subject with a glioma can be effected by a method comprising administering to the subject an acridine analog or a pharmaceutically acceptable salt or hydrate thereof in connection with radiation therapy. For example, inhibiting intracranial metastasis of gliomal cancer cells in a subject can be effected by a method comprising administering to the subject an acridine analog or a pharmaceutically acceptable salt or hydrate thereof in connection with radiation therapy. For example, preventing relapse of glioma in a subject can be effected by a method comprising administering to the subject an acridine analog or a pharmaceutically acceptable salt or hydrate thereof in connection with radiation therapy.

While not wishing to be bound by theory, it is believed that acridine analogs, in combination with radiotherapy, can have an augmented or synergystic effect on the subject. Further, acridine analogs, when used in combination with radiotherapy, can lower a subject's need for radiotherapy (e.g., less radiation need be used) and/or can lower a subject's need for acridine analogs (e.g., acridine analogs can be given in dosages lower than would be typically adminstered as single-agent therapies).

It is also understood that the disclosed acridine analogs, when adminstered to subjects in connection with radiation therapy, can also be employed in combination with one or more chemotherapeutic drugs and/or in connection surgical therapy.

7. Use in Connection with Surgical Treatment

Surgery can be used to treat gliomas. In one aspect, the invention relates to the administration of the disclosed acridine analogs to subjects in connection with surgical treatment. For example, treating a subject with a glioma can be effected by a method comprising administering to the subject an acridine analog or a pharmaceutically acceptable salt or hydrate thereof in connection with surgery. For example, inhibiting intracranial metastasis of gliomal cancer cells in a subject can be effected by a method comprising administering to the subject an acridine analog or a pharmaceutically acceptable salt or hydrate thereof in connection with surgery. For example, preventing relapse of glioma in a subject can be effected by a method comprising administering to the subject an acridine analog or a pharmaceutically acceptable salt or hydrate thereof in connection with surgery.

It is contemplated that acridine analogs can be adminstered before, during, or after surgical treatment. While not wishing to be bound by theory, it is believed that acridine analogs, in combination with surgery, can have an augmented or synergystic effect on the subject. Further, acridine analogs, when used in combination with surgery, can lower a subject's need for surgery (e.g., less tissue need be removed) and/or can lower a subject's need for acridine analogs (e.g., acridine analogs can be given in dosages lower than would be typically adminstered as single-agent therapies).

It is also understood that the disclosed acridine analogs, when adminstered to subjects in connection with surgical therapy, can also be employed in connection with radiation therapy and/or surgical therapy.

8. Use in Preventing of Relapse of Glioma

The disclosed compositions can also be employed to prevent relapse in a subject previously treated for a glioma. In one aspect, such a method comprises administering to the subject a prophylactically effective amount of an acridine analog or a pharmaceutically acceptable salt or hydrate thereof. It is understood that the dosage needed to prevent relapse (i.e. maintenance dose) may be less (e.g., half) of the dosage needed to effect treatment of a glioma. Thus, in maintenance, a suitable dosage of the acridine analog or a pharmaceutically acceptable salt or hydrate thereof can be from 0.5 to about 250 mg/kg of the subject, can be administered at a dosage of from 0.05 to about 100 mg/kg of the subject, can be administered at a dosage of from 0.05 to about 50 mg/kg of the subject, or can be administered at a dosage of from 0.01 to about 250 mg/kg of the subject.

It is also understood that when using the disclosed acridine analogs for preventing of relapse of glioma, in either single agent therapy or in combination therapy, can be also adminstered to subjects in connection with surgical therapy and/or surgical therapy.

K. Compositions with Similar Functions

It is understood that the compositions, such as quinacrine and the acridine analogs, disclosed herein have certain functions, such as antimetastatic activities or anti-proliferative activities. Disclosed herein are certain structural requirements for performing the disclosed functions, and it is understood that there are a variety of structures which can perform the same function which are related to the disclosed structures, and that these structures will ultimately achieve the same result, for example, inhibition of anti-proliferative activities.

L. Processes for Making the Compositions

Disclosed are processes for making the compositions as well as making the intermediates leading to the compositions. There are a variety of methods that can be used for making these compositions, such as synthetic chemical methods and standard molecular biology methods. Generally, however, the acridine analogs disclosed herein are commercially available or can be made my methods known in the art. The peptides disclosed herein can be isolated from natural sources by methods known or can be prepared by known peptide synthetis routes.

### M. MANUFACTURE OF A MEDICAMENT

In one aspect, the invention relates to a method for the manufacture of a medicament for treating or preventing a disease of uncontrolled cellular proliferation (e.g., glioma) in a subject (e.g., mammal) comprising combining a therapeutically effective amount of a disclosed compound or product of a disclosed method with a pharmaceutically acceptable carrier or diluent.

### N. KITS

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits can include molecules, including for example, quinacrine or acridine analogs, for use in vitro cell assays as standards for anti-proliferative activity.

In various aspects, the invention relates to a kit comprising a disclosed compound or a product of a disclosed method and one or more of at least one agent known to increase risk of glioma; at least one agent known to decrease risk of glioma; at least one agent known to treat glioma; at least one agent known to treat a disease of uncontrolled cellular proliferation; an/or instructions for treating a disease of uncontrolled cellular proliferation. In further aspects, the at least one compound or the at least one product and the at least one agent are co-formulated. In a further aspect, the at least one compound or the at least one product and the at least one agent are co-packaged.

### O. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

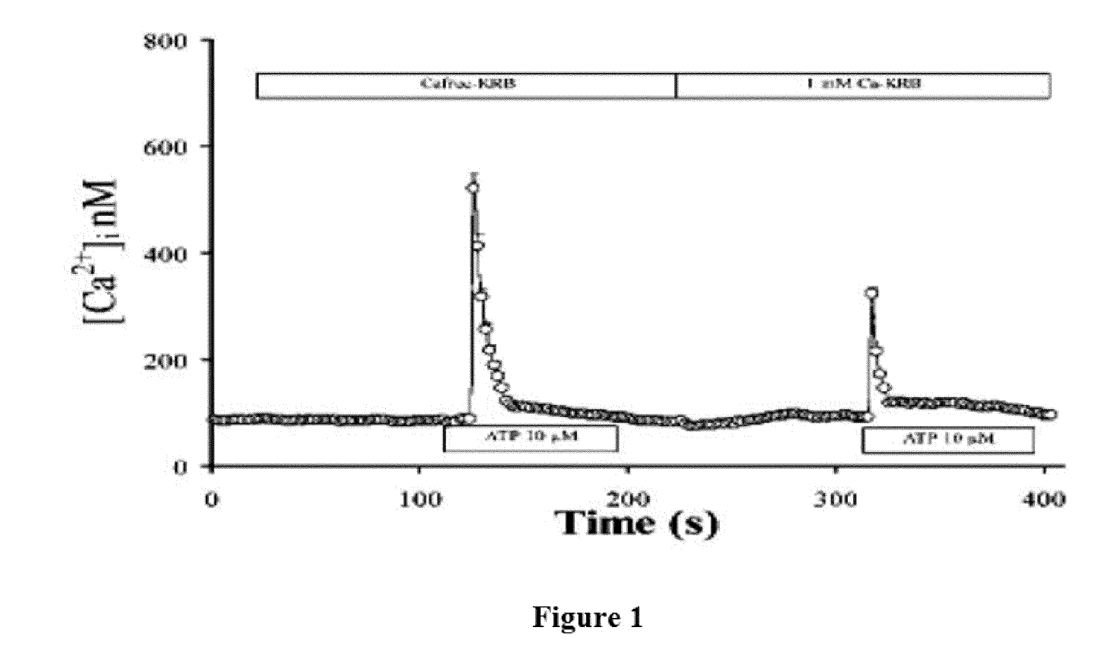

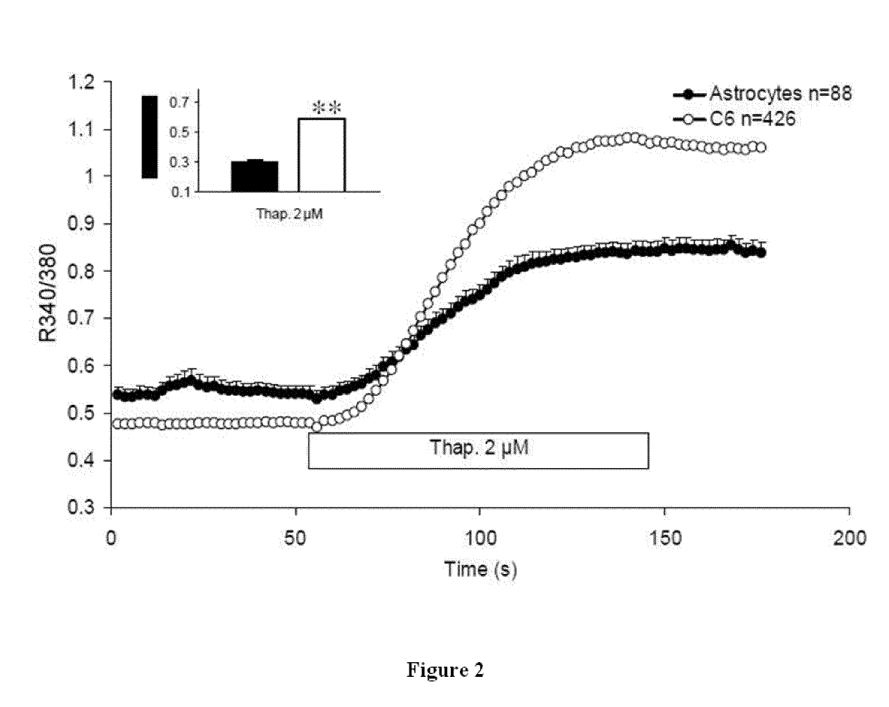

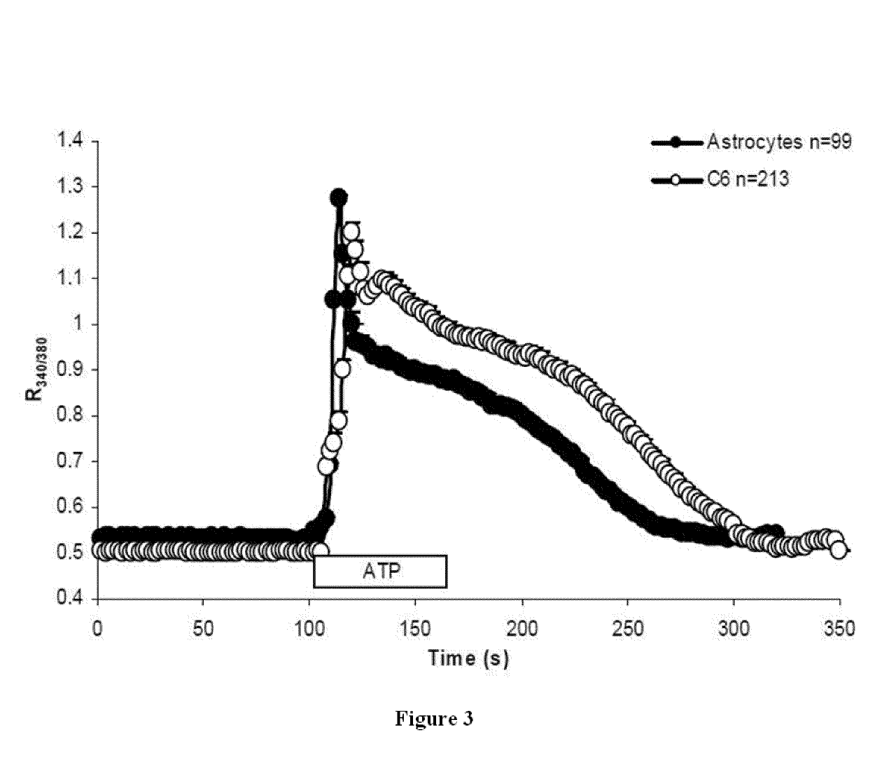

Tools and protocols have been developed to cleanly observe capacitative Ca²⁺ entry (CCE), the phenomenon that allows the replenishment of intracellular Ca²⁺ (ICS) stores and make quantitative and qualitative assessments. In particular, in astrocytes, capacitative Ca²⁺ entry happens in absence of cytoplasmic Ca²⁺ concentration ([Ca²⁺]ᵢ) elevation (Grimaldi; 2006)(FIG. 1). As previously demonstrated, this particular ability of astrocytes is related to SERCA function and expression. In fact, this peculiar, if not unique, behavior of astrocytes was shown to be a functional rather than a structural ability. Investigation of this phenomenon revealed that SERCA is the key player in the dynamic equilibrium, conferring astrocytes the ability to refill intracellular Ca²⁺ stores very efficiently and in the absence of [Ca²⁺]ᵢ elevation (Grimaldi; 2006). The study was designed to investigate if there was a difference between astrocytes and glial cancer cells in this distinctive homeostatic pattern. The findings presented in this study, indicate that there are striking differences between glial cells and their cancer counterpart. The model utilizes rat cells but the observations extend and apply to human glioma cells as well. When the responses of normal astrocytes and C6 cells to thapsigargin (THAP), an agent able to block SERCA, therefore causing the release of Ca²⁺ from the ER, were compared it was evident that C6 was more sensitive to the THAP (FIG. 2). However, the difference can be ascribed to one of many possibilities. Since THAP is not a physiological signaling factor, studies were conducted in order to verify that this response is also present during physiological agonist stimulation. Therefore the experiment was repeated using ATP, an important signaling molecule for astrocytes, which is released by neurons and astrocytes themselves and is involved in synchronization of astrocytes neurons and astrocytes-astrocytes interaction. Results from this experiment provided evidence that C6 responded to ATP with an increased elevation of [Ca²⁺]ᵢ (FIG. 3). However, the increase of [Ca²⁺]ᵢ elevation in C6 mostly affected the plateau phase rather than the peak phase.

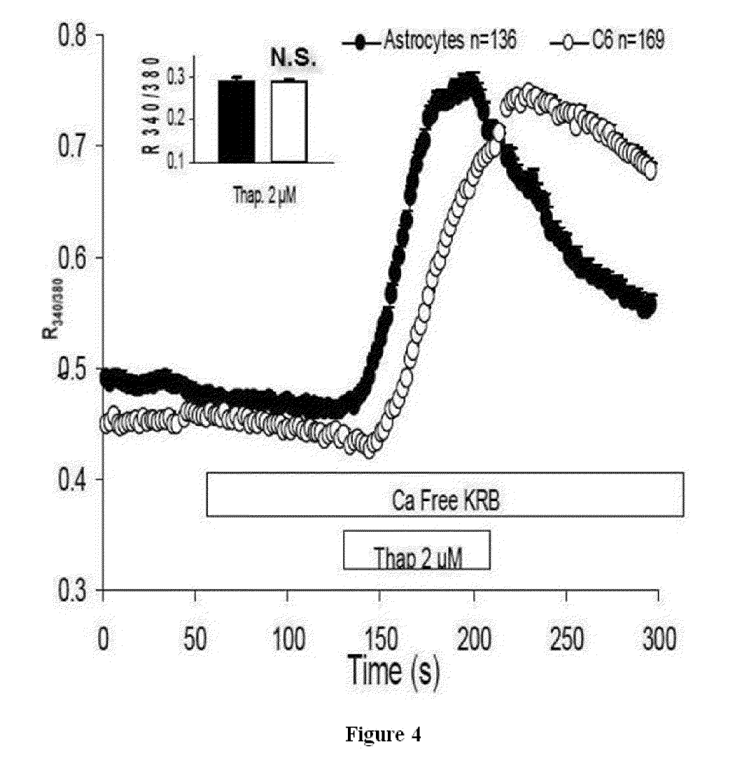

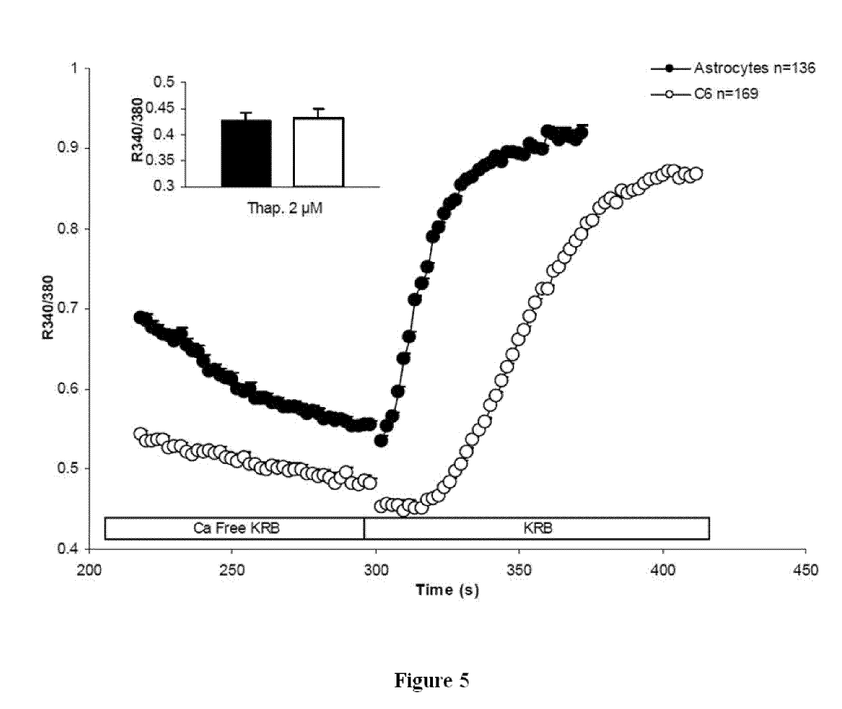

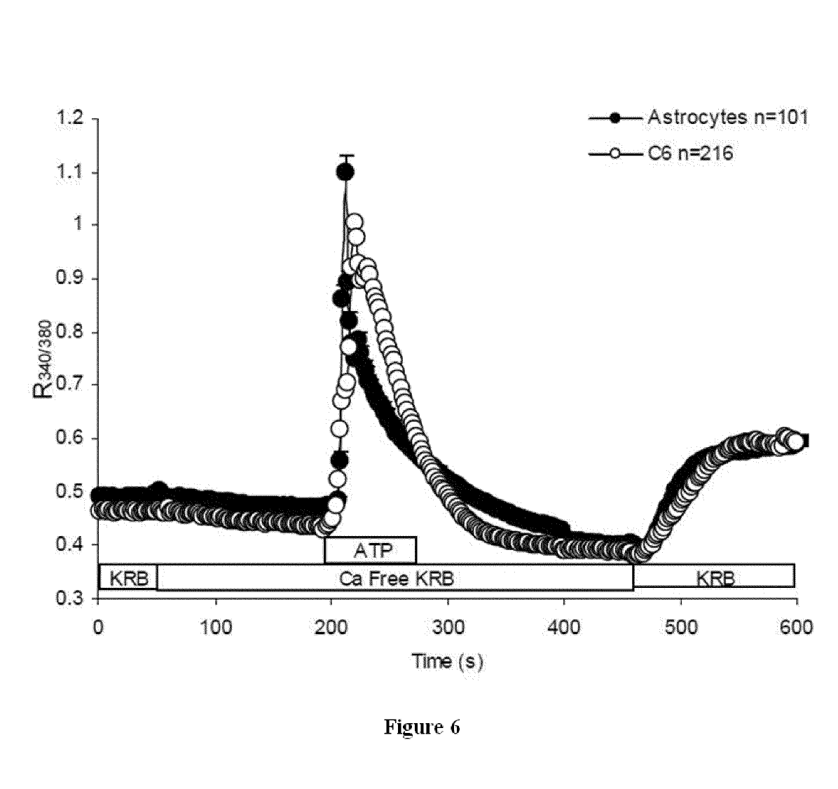

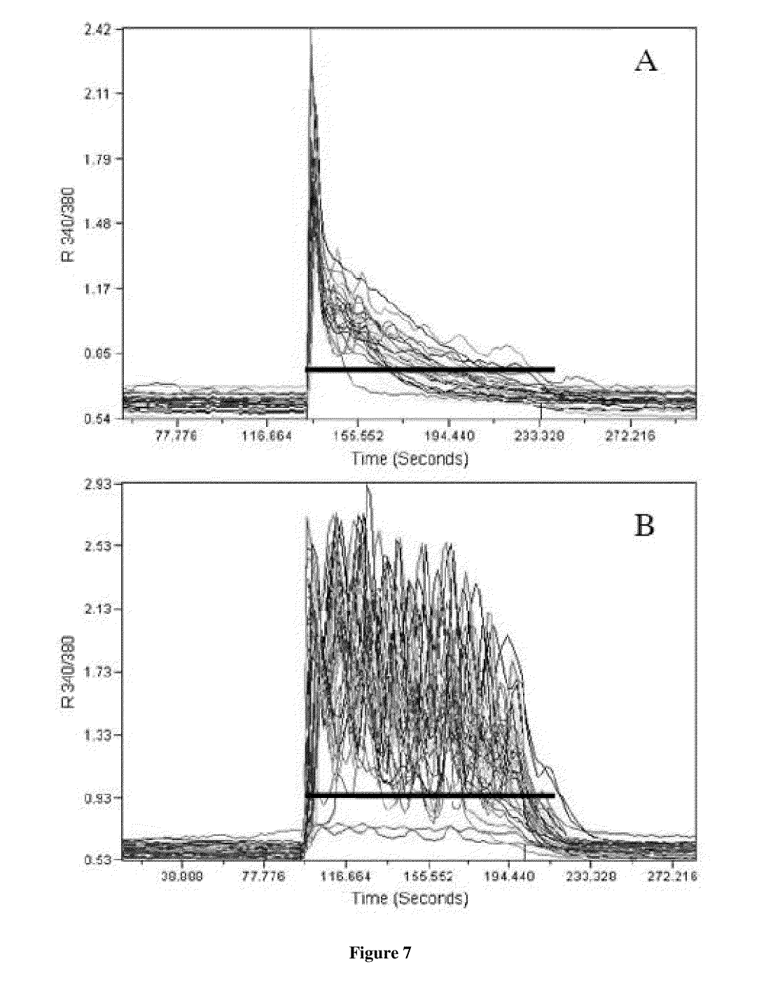

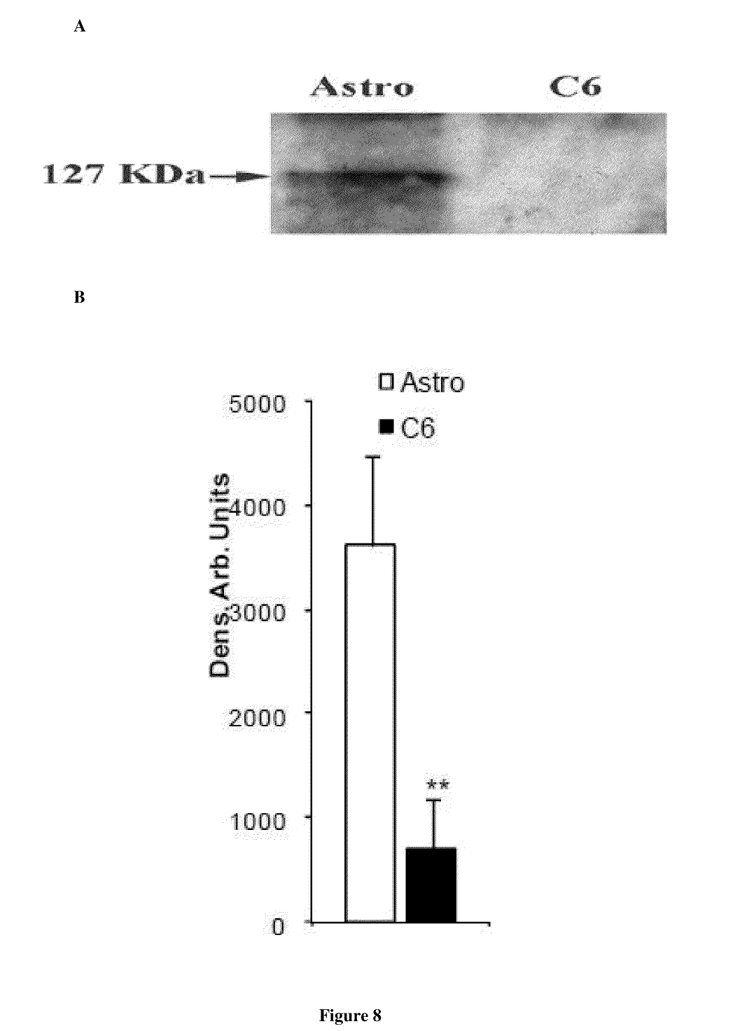

This phase is dependent on the balance of Ca²⁺ influx triggered by store depletion and the reuptake in the ER due to SERCA activity. Hence, this specific part of the Ca²⁺ transient response was investigated to see what was causing C6 to have larger responses. When astrocytes and C6 were challenged with THAP in the absence of extracellular Ca²⁺, responses were completely identical indicating that the amount of Ca²⁺ stored in the ER are about the same in the two cell types (see FIG. 4). To investigate the role of influx triggered by ICS depletion, CCE was analyzed following THAP-induced depletion of ICS. In these conditions, initiation of influx by addition of Ca²⁺ produced absolutely identical responses in the two cell types. These data represent strong evidence that the differences observed are not due to the level of the influx of Ca²⁺ (FIG. 5). Similar data were produced using ATP (see FIG. 6). However, ATP experiments, in both cases displayed in FIGS. 2 and 6, indicates a larger plateau phases in C6. Previous experiments addressing the role of extrusion mechanisms on the shape of Ca²⁺ transients in astrocytes have concluded that inhibition of the plasma membrane Ca²⁺ pumps did not affect transients significantly. Other abnormalities were disclosed by analyzing the Ca²⁺ transients elicited in cells exposed to tunicamycin (TUN). In particular, TUN pretreatment caused the appearance of [Ca²⁺]ᵢ oscillations at high frequency and high amplitude in C6, while transients were not changed in astrocytes. The source of Ca²⁺ for these oscillation triggered by TUN was entirely intracellular indicating that C6 cells reacts to induction of ERSR with a large timed and repetitive release and uptake of calcium from the ER in response to physiological stimuli such as ATP, a common neurotransmitter (FIG. 7). All these data indicate a dysfunction in C6 pertaining the uptake of Ca²⁺ in the ER via SERCA. Assessment of the level of SERCA in astrocytes and C6 was able to consistently show, via immunoprecipitation followed by western blot with two different commercially available SERCA antibodies, that SERCA levels are lower in C6 than in astrocytes (FIG. 8). There are a number of cellular phenomenons that take place in the ER or see the ER as a central organelle that can be affected by the dysfunctions highlighted in the previous experiments. However, a clear link between ER Ca²⁺ storage deficit and physiological phenomenon that can be relevant for disease development is not clearly identified. Moreover, scarce data are present in literature about dynamic Ca²⁺ deregulation and disease mechanisms.

### 2. Example 2

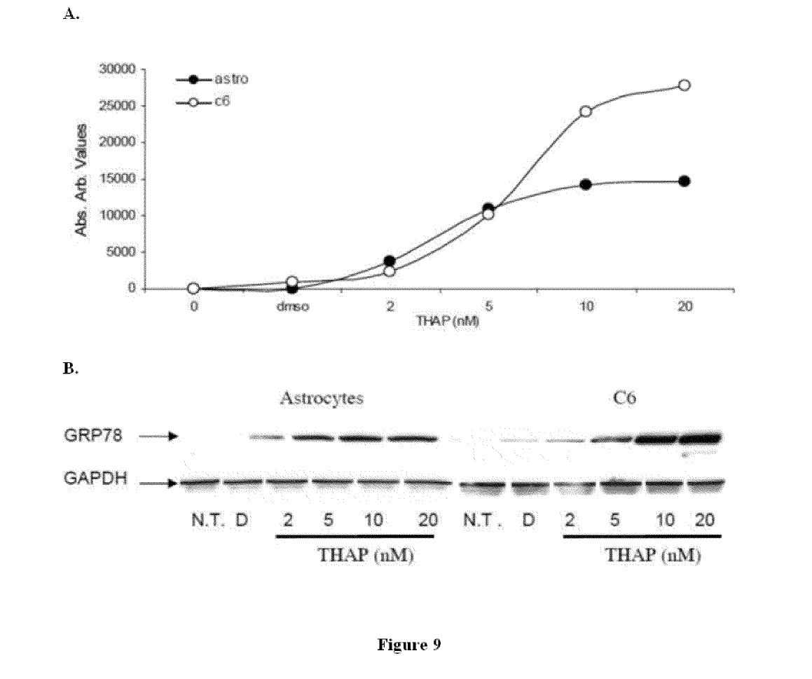

SERCA is one of the proteins that is up-regulated during the ER stress induction by GRP78, being part of the adaptive response to faltering protein folding in the ER. Therefore, experiments were conducted in order to determine if the abnormality in Ca²⁺ homeostasis and reduced SERCA expression in glioma cells were responsible for their behavior. As such, GRP78 regulation in astrocytes and C6 was initially analyzed during ERSR induced by THAP. Both cell types increased GRP78 expression in response to THAP similarly up to the EC₅₀ of the substance (2-5 nM). Higher THAP concentrations caused a dramatically higher elevation of GRP78 in C6 (FIG. 9). These findings are compatible with findings that lower concentrations of THAP do not inhibit SERCA maximally, therefore some compensation can occur. However, in C6, in which there is less SERCA, compensatory mechanisms are overwhelmed earlier than in astrocytes. Hence, the dramatic increased induction of GRP78 in C6 (FIG. 9) due to lowered Ca²⁺ in the ER. These data fit perfectly with the prediction that C6, due to their lower level of SERCA, is more sensitive to Ca²⁺-depletion induced ERSR, because they are not able to reuptake Ca²⁺ in the ER as well as astrocytes in order to prevent protein misfolding.

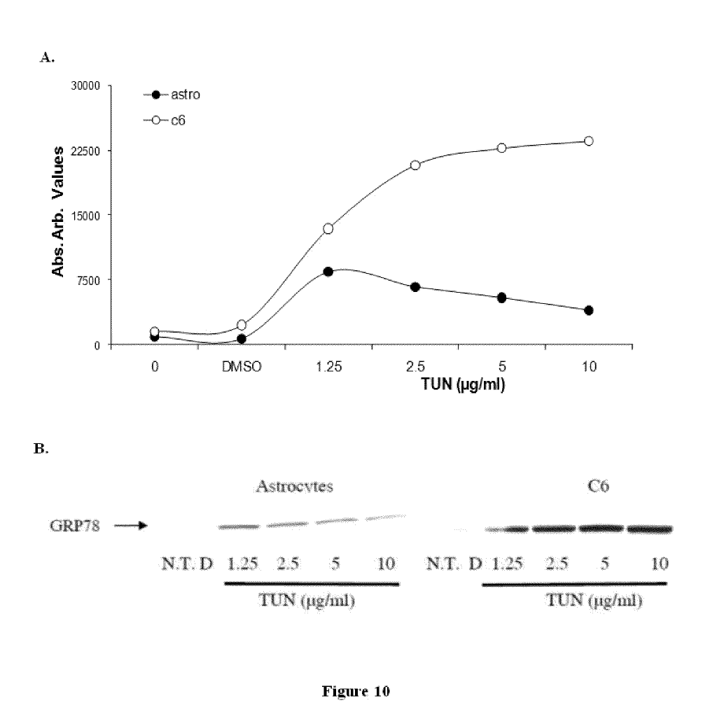

Since Ca²⁺ represents an ionic chaperone for protein folding in the ER, it is presumable that in conditions in which there is a limited amount of the ion in the ER to help with protein folding, other agents affecting ERSR such as TUN, which causes protein misfolding by preventing N- and O-glycosylation of novel proteins, be affected as well. Exposure to TUN caused a concentration related elevation of GRP78 protein expression in both astrocytes and C6. However, as with THAP, the up regulation of GRP78 was significantly larger in C6, indicating the struggle of these cells to cope with ERSR. In particular, TUN effect was larger in C6 than in astrocytes at every concentration tested (FIG. 10). Experiments were then conducted to determine if this enhanced sensitivity of C6, a rat astrocytoma cell line, was just a feature of these cells or was also shared by human gliomas or astrocytomas.

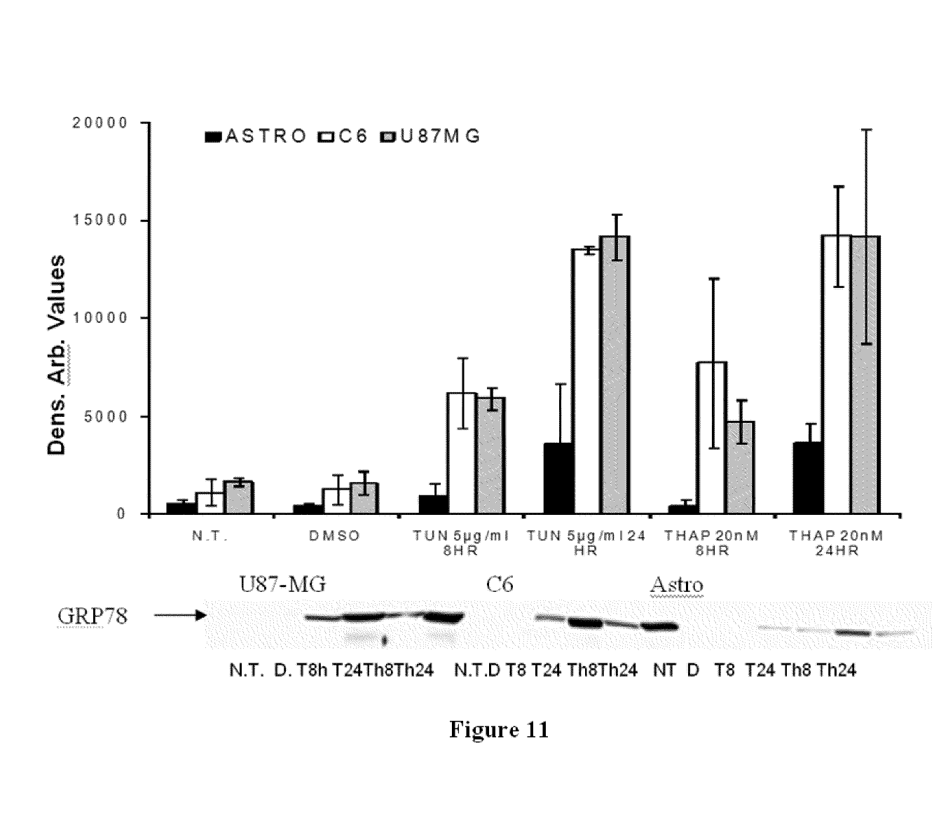

Also it has been speculated that the increased higher constitutive expression in glioma cells determines their resilience to the hostile growth environment, to pharmacological treatment and to radiotherapy. However, these studies were performed comparing human astrocytes from repositories to several human malignant cell lines both commercially available or from patients. Experiments have been performed using these human astrocytes from repositories and they do not share several of the signaling features of primary astrocytes from rat and of several human glioma cell lines, which represents known feature of astrocytes in vivo. Also, in these studies, it was concluded that pharmacological up regulation of GRP78 in glioma cells resulted in reduction of proliferation and growth, and ultimately killed glioma cells. These data were therefore in disagreement in that if elevated GRP78 levels were protective, further increase resulted in more protection. Therefore, in light of the preliminary observations, experiments were conducted in order to further investigate the phenomenon with the aim to better understand it. Experiments were performed using primary rat astrocytes and their glioma counterpart, the C6 cells; also, human glioma cells, such as U87-MG, were used. The latter have been widely used in assessing human glioma properties. Unstimulated expression levels of GRP78 were compared in these cells. In FIG. 11 it is possible to view that unstimulated GRP78 expression in these three cell types is very low. Expression of GRP78 can be enhanced significantly by both exposure to THAP or TUN, following a fairly brief exposure (8 h). However, astrocytes GRP78 levels following activation of ERSR with the indicated agents were always significantly lower than levels reached in C6 and U87-MG. Therefore, from this data, it appears that rather than an elevation of resting GRP78 expression in these cells, it is present hyperactivity of ERSR as signaled by higher level of GRP78 induction.

### 3. Example 3

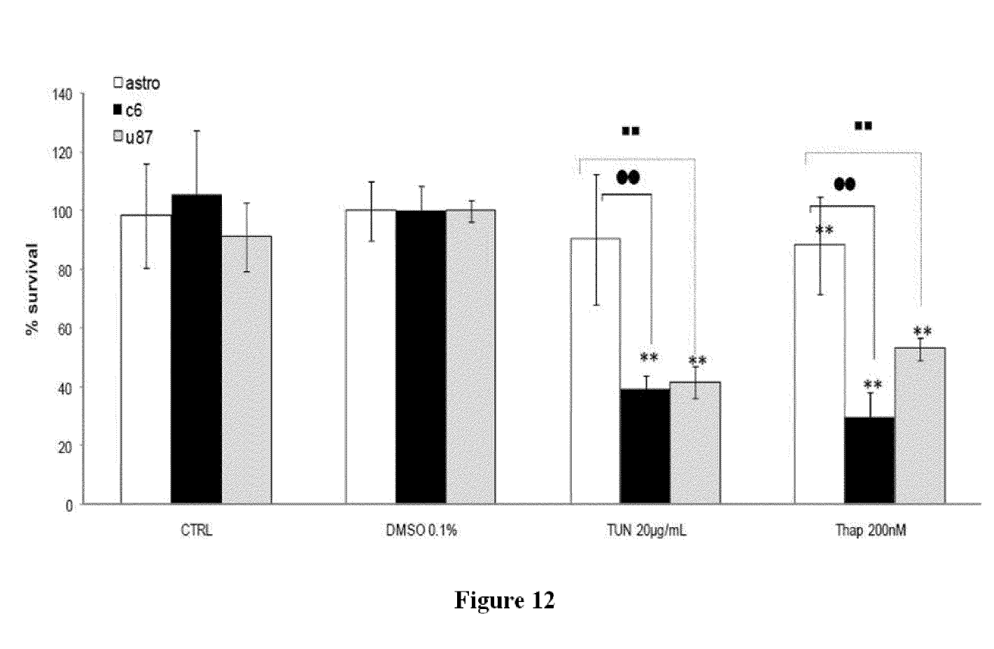

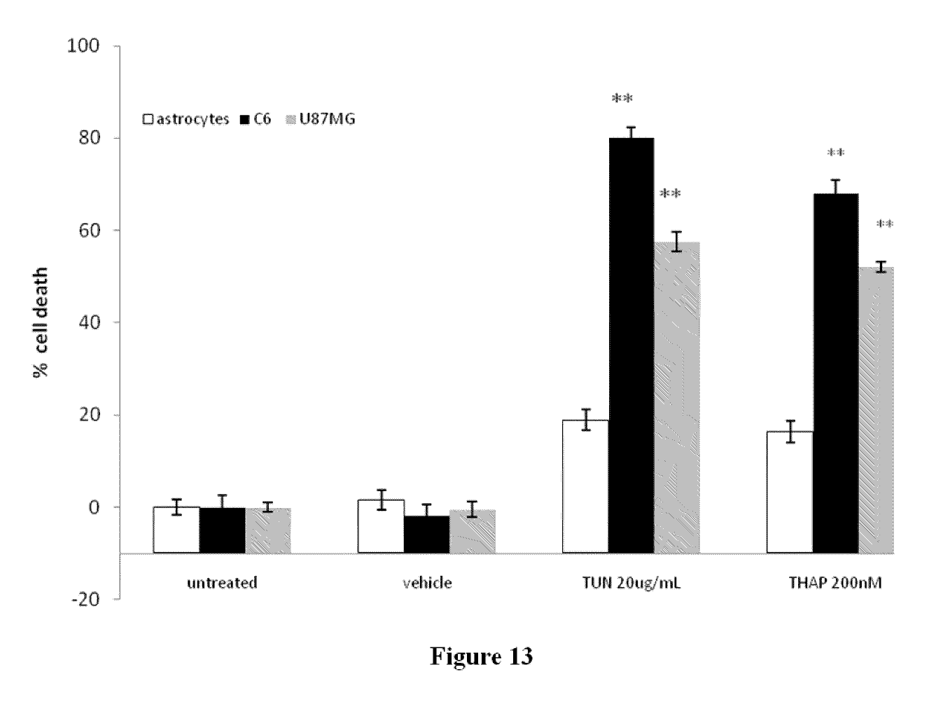

These results show a number of critical differences between normal cells and glioma cells, both rat and human, indicating hypersensitivity to ERSR inducing agents. In order to verify how the hypersensitivity of this signaling pathway in glioma cells affects the survival of the cells, viability assessments were performed using at least three unrelated biochemical tests. Initially, a commercially available kit (Cell Titer Glow) was used in the high throughput screening center for cytotoxicity screening. This test is amenable to high throughput screening techniques and, therefore, can be used for follow up of selected compounds in high or medium throughput settings. This kit assesses intracellular content of ATP as an indicator of cell well being revealed as luciferase activity. FIG. 12 shows the effect of ERSR activation on astrocytes, C6 and U87-MG using this commercially available cytotoxicity kit. C6 and U87-MG display significantly higher cytotoxicity, than astrocytes, when challenged with THAP and TUN for 48 hours. Since, THAP inhibits SERCA, an ATPase, and it can affect ATP levels directly the effect of THAP and TUN on these 3 cells types was also assessed using a commercially available kit that assesses metatetrazolium salts conversion (XTT). XTT assesses cell viability by probing mitochondrial integrity. After 24 hours of treatment with the indicated agents, C6 showed a significantly higher inhibition of XTT conversion than astrocytes. Longer incubation times (48 h) resulted in a stronger inhibition. Finally, viability of U87-MG was also assessed using a system measuring the activity of a house keeping cytosolic enzyme involved in glucose metabolism. Also using this detection system it was evident that glioma cells both C6 and U87-MG were greatly affected by induction of ERSR (FIG. 13). It is well known that prolonged ERSR leads to apoptosis, even in the presence of elevated GRP78 expression if that is coupled to significant protein unfolding. In rat cells this process is actuated via caspase 12 and ultimately via caspase 3 activation. Experiments were conducted in order to determine if the observed effect on cell viability was also paralleled by caspase 12 activation, and if there were differences, as indicated by preliminary viability data, between normal and glioma cells.

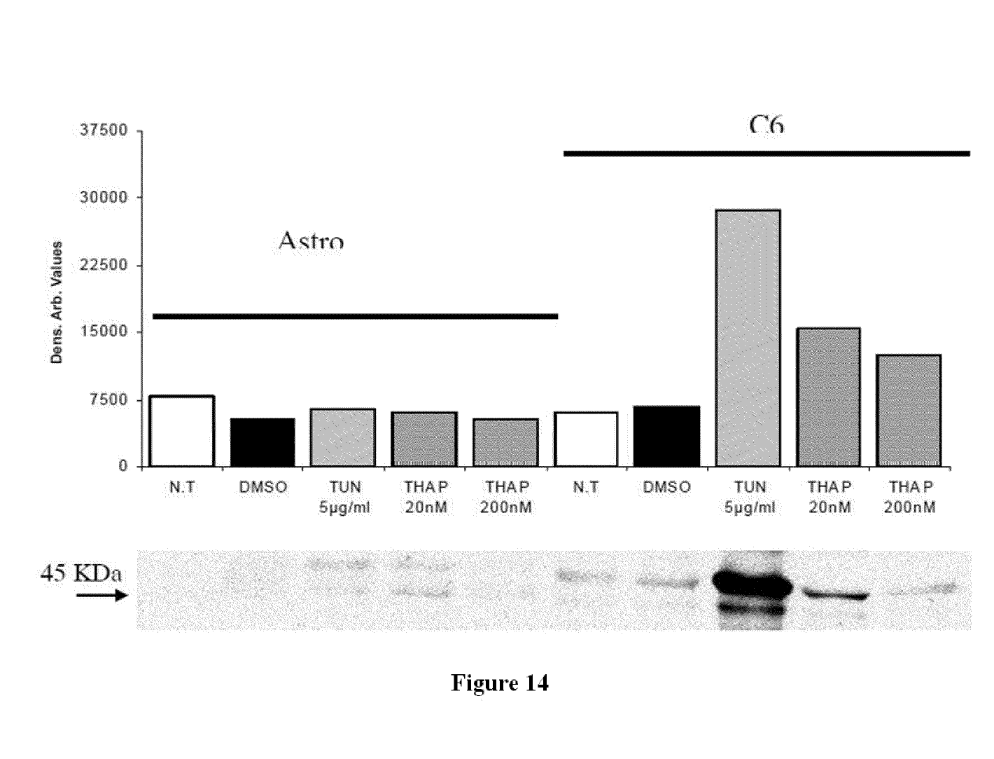

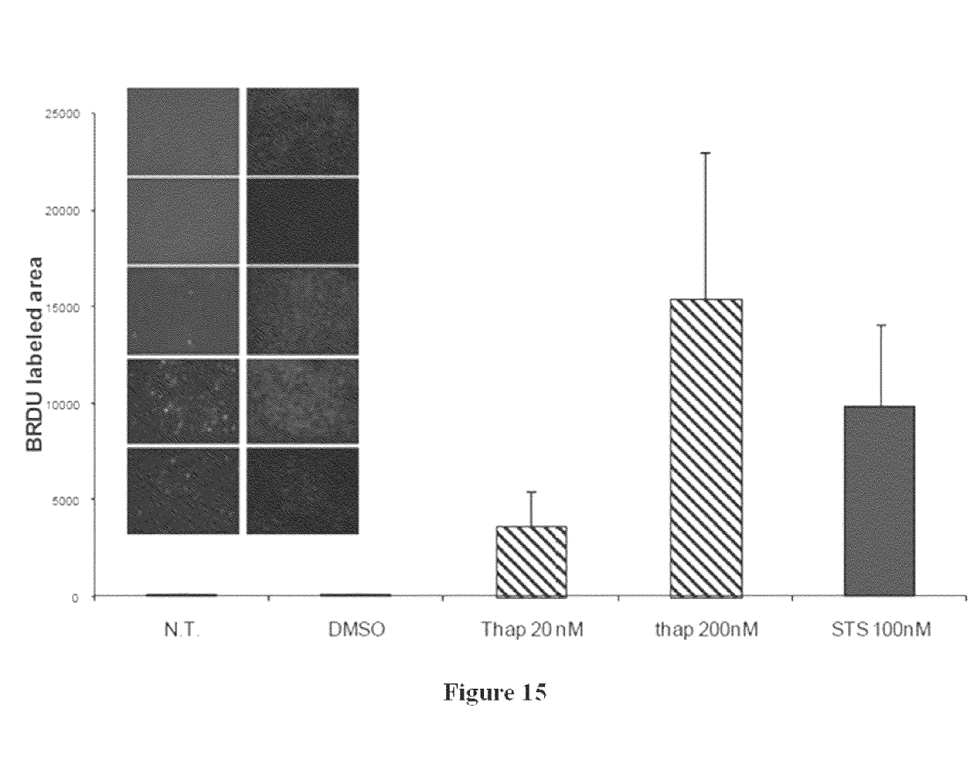

Cells were treated with the different agents as indicated in FIG. 14. Thereafter, cells were harvested and protein extracted. Procaspase 12 band at 65 kDa was identified. In some of the treatments, the active product at 45 kDa was also detected. In particular, in C6 a significant activation of caspase 12 was detected following treatment with TUN and THAP. Astrocytes were completely unresponsive. These preliminary data indicate that, in astrocytes, although there is a measurable up-regulation of GRP78, either its levels or the conditions are not conducive to caspase activation. Conversely, in C6, higher GRP78 levels and the cell environment reflect significant caspase activation. Additionally, astrocytes can possess a mechanism of action that repress caspase activation, such as NF-κB activation, which is a classical component of ERSR that is also activated by Ca²⁺ elevation in the cytoplasm in cells treated with THAP. Caspase activation in ERSR conditions was also confirmed with a TUNEL based apoptosis detection system in C6. C6 were treated with THAP for 24 hours and thereafter processed for TUNEL accordingly to manufacturer instructions. Staurosporin, a well known inducer of apoptosis in glial cells, was also included as a positive control. High resolution images of the experiments were acquired and image morphometric analysis was performed to determine quantitative parameters of the staining as previously described (Pascale et al., 2004, “Translocation of protein kinase C-betaII in astrocytes requires organized actin cytoskeleton and is not accompanied by synchronous RACK1 relocation.” Glia 46(2): 169-82). The results show a clear increase of TUNEL labeling in cells treated with THAP and staurosporin (FIG. 15). At this point the data indicated that apparently in glioma cells there is a heightened sensitivity to induction of ERSR that can be monitored by and parallels very well with GRP78 induction. Also, the induction of ERSR and GRP78 in glioma cells both human and rat is followed by cell death that is much lower in their normal counterpart.

### 4. Example 4

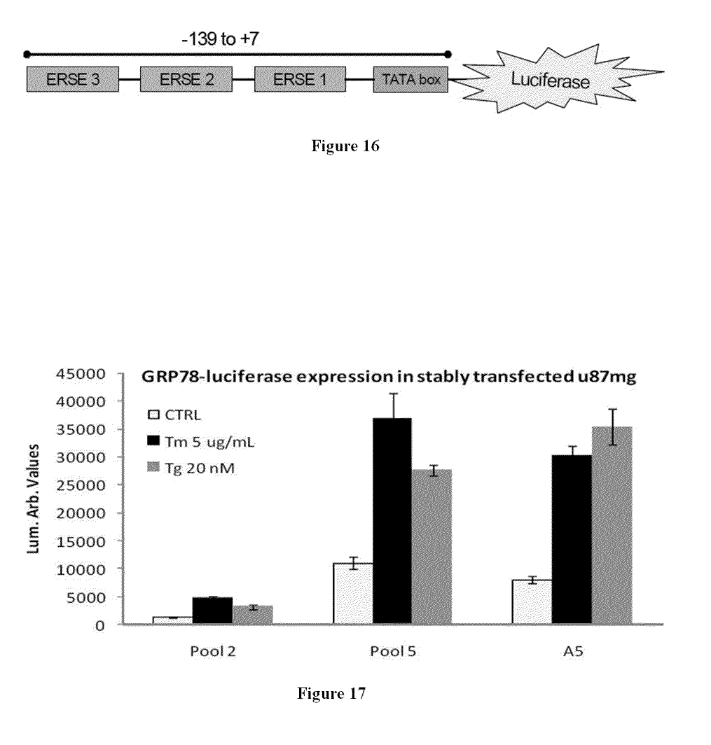

A construct coding a large part of the human GRP78 promoter, attached to the reporter gene firefly luciferase was used (see FIG. 16 for a schematic) (Yoshida et al., 1998).

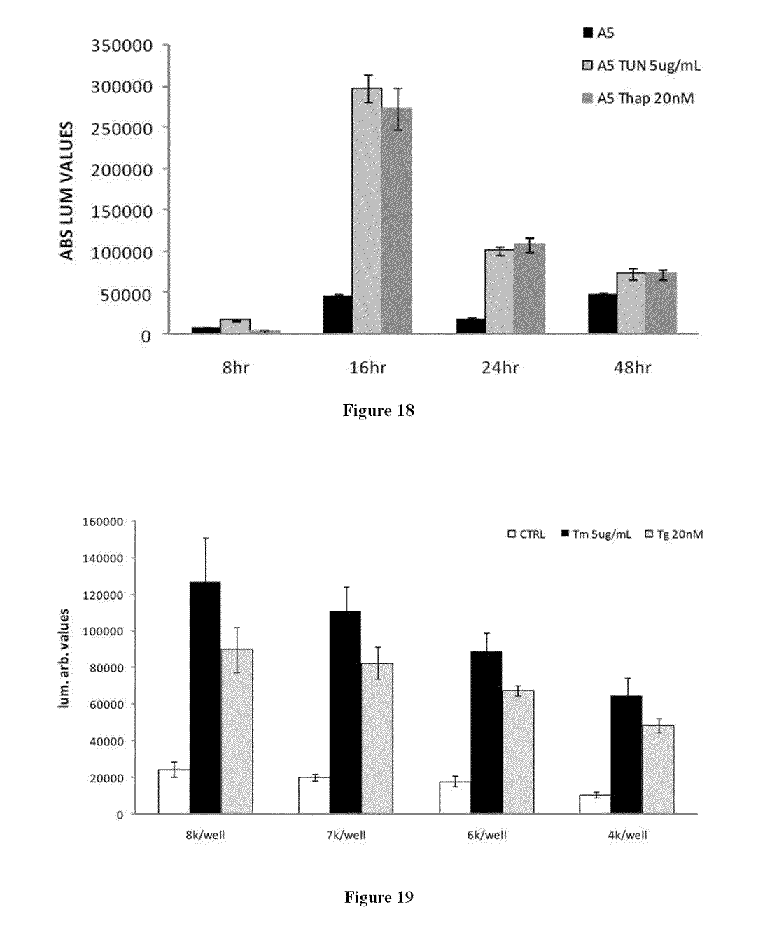

The promoter region contains the sequence of the GRP78 promoter from −139 to +7. In this part of the promoter there are three ER stress responsive elements and the TATA box to which is linked in frame the firefly luciferase sequence (FIG. 16)(Yoshida et al., 1998). The construct, which was cloned in an expression vector, was transfected into E. Coli, amplified, purified and checked it by enzymatic digestion. Thereafter, C6 and U87-MG were transiently transfected with the plasmid. After 24 hours the cells were challenged with THAP and TUN for 24 hours. Although, in transient transfection only a small part of the cell population expresses the transduced gene, a significant induction by THAP and TUN was detected over 24 hours. Armed with these encouraging data human glioma cells U87-MG were transfected with both the above described construct and a construct carrying resistance to blasticidin. The sensitivity of these cells to the antibiotic has previously been assessed. 24 hours after the transfection the antibiotic at its 75% inhibitory concentration was added. The cells were given time to proliferate. Two pools called P2 and P5 were selected and luciferase expression was assessed. Both Pool 2 and pool 5 expressed unstimulated Luc signal, which was greatly enhanced by exposure to THAP and TUN. Clones from these two pools were isolated. Luciferase expression in the clones was characterized and a clone called A5, in which baseline expression of the construct was reasonably low and induction with two positive controls was large and consistent, was selected. This clone, several back up clones, and the pools were amplified and frozen in sufficient amounts to guarantee continuity in case needed. Newly thawed cells were used to prepare the summarized data that are presented in FIG. 17-21. Next, the effect of the incubation time on luciferase induction was assessed. Since this assay can be affected by false negative due to toxicity of hit compounds, the incubation times were kept as short as possible. Therefore, several incubation times were tested, including 8, 16 and 24 h. The goal was not to incubate longer than 24 hours because toxicity starts to develop at that time point. FIG. 18 shows the data for these time courses. The data indicate that the optimal incubation time for this assay is 16 hours. At this time point the induction is about 6-7 fold. Previously, using a different assay, a half a million compound library was screened very easily using 2.5 thousands cells per well in 1536 plates. FIG. 19 shows the effect of various cell densities on the assay performance in half area 96 well plates (these are only double the size of a 384 well plate). With this cell number the dynamic range is large and the fold of induction of 6-7 is maintained.

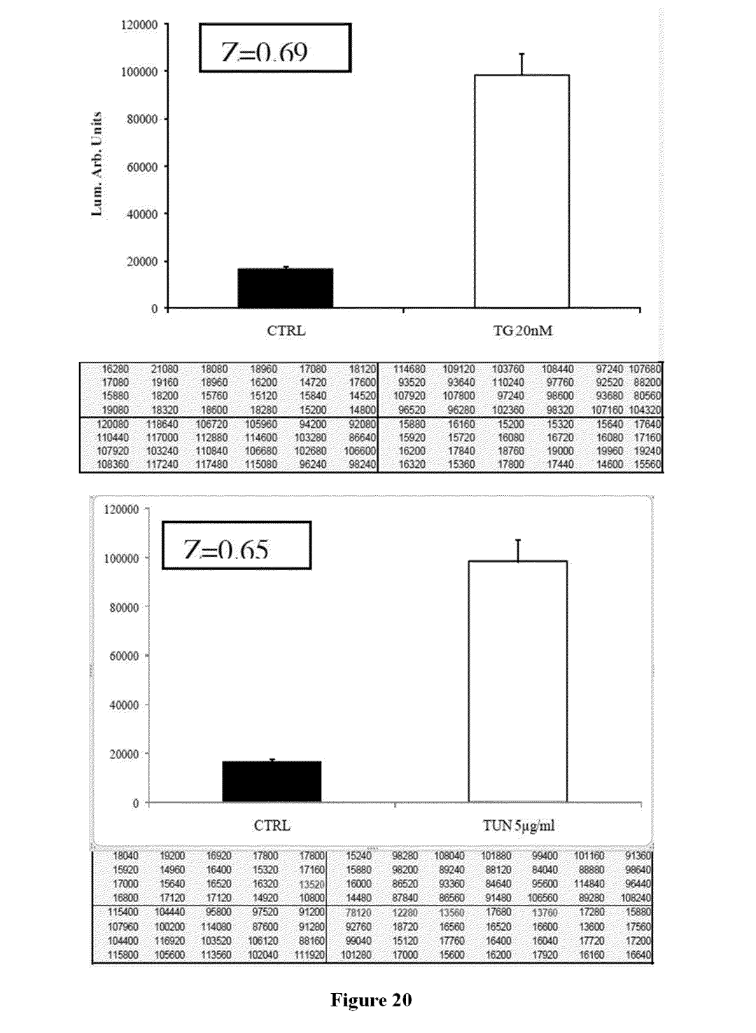

It is common practice in the field of high throughput screening to assess the so called z number of the assay. This statistical parameter indicates the suitability of a given assay for high throughput screening. The z-value in a word describes the likelihood of a single point hit not to be false positive. The z value is a number lower than or equal to one. Values above 0.5 indicate a robust assay. Values below 0.5 but above 0 indicate an assay that probably needs to be run in duplicate. Negative values indicate assays whose predictability is very low. Z values are usually determined in a high density format plate usually a 96 well plate in manual conditions or a 384-1536 plate format in automated liquid handling environment. Z value determination has been performed using half area 96 well plates in manual mode. FIG. 20 shows the behavior of the assay in a standard inverted quadrant Z plate. The positive controls TUN and THAP, which have been extensively characterized in this system (see above), were very effective and gave about 6-7 fold induction in a very reproducible manner. Z values were very high at 0.65 or above (FIG. 20).

5. Example 5

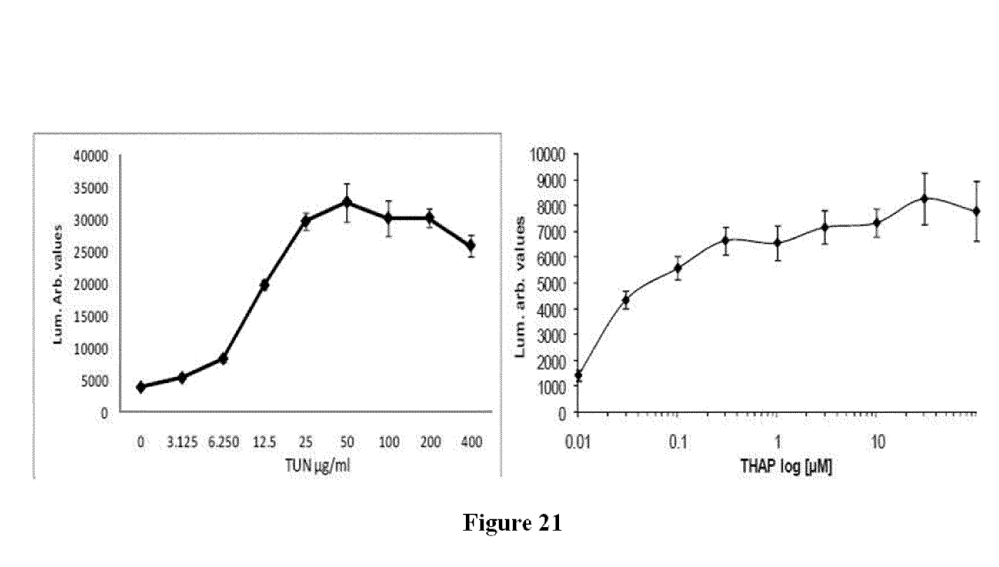

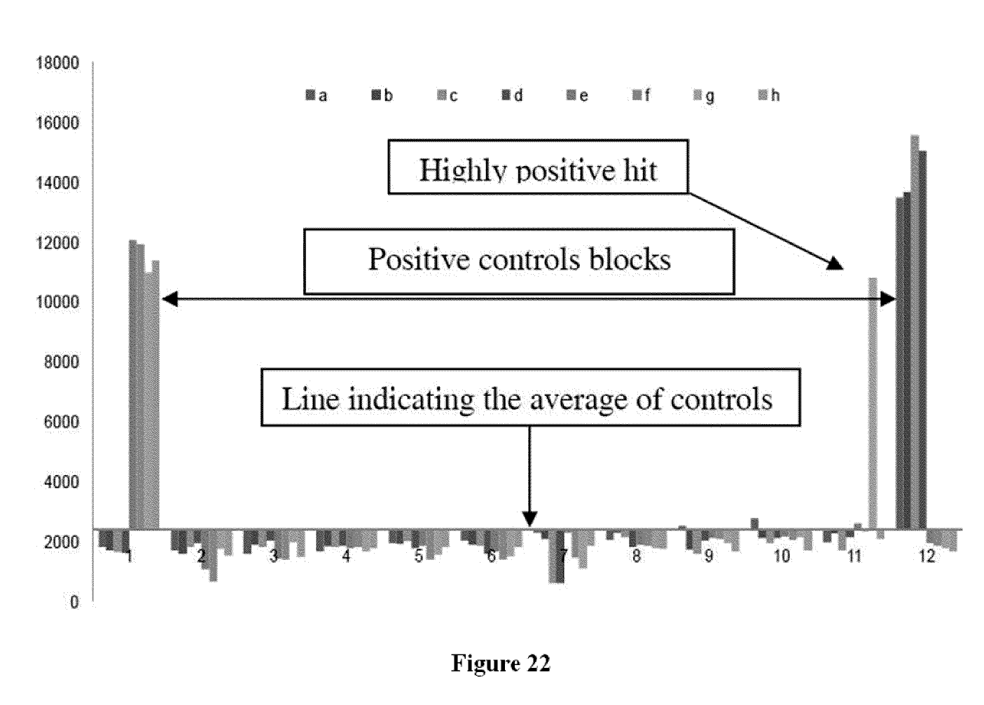

The assay was performed in order to determine the behavior of the two positive controls and the sensitivity to DMSO, since most of the libraries are resolved in DMSO. FIG. 21 shows the concentration profile of TUN and THAP in AS cells as far as induction of GRP78 driven luciferase. Both agents elicited an increase of luciferase expression driven by GRP78 promoter compatible with their induction of GRP78 native protein in U87-MG (parental cell line) and their pharmacological properties inherent to the inhibition of SERCA, in the case of THAP, and glycosylation inhibition in the case of TUN. Therefore, if any hits are picked up, they are relevant probes for this pathway. The sensitivity of the cell line and of their response to DMSO was also characterized. The data indicates that up to 0.5% DMSO does not affect either survival of AS cells or their response to the positive controls. Previous studies highlighted the path to screening with an initial phase of validation including a screening of a small molecule repository such as the Prestwick compound collection, which includes almost all of the FDA approved drugs on the market plus several hundred natural product molecules. This has been accomplished in manual mode. The Prestwick repository was actually screened with this assay using automated drugging of the plate and manual cell seeding and reading. The screening was accomplished in one session. An example of the results obtained in one plate is shown in FIG. 22. The statistical analysis of the data indicated that compounds scoring a value higher than the average of the negative controls plus 3 standard deviations (SD) were highly likely to be compounds of interest for follow up.

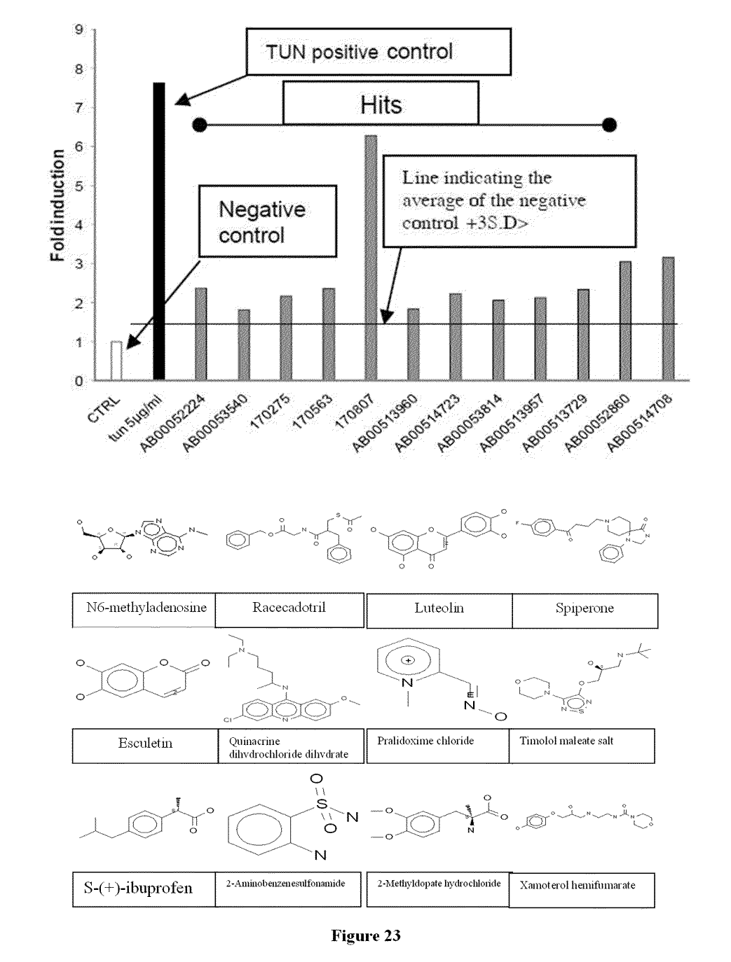

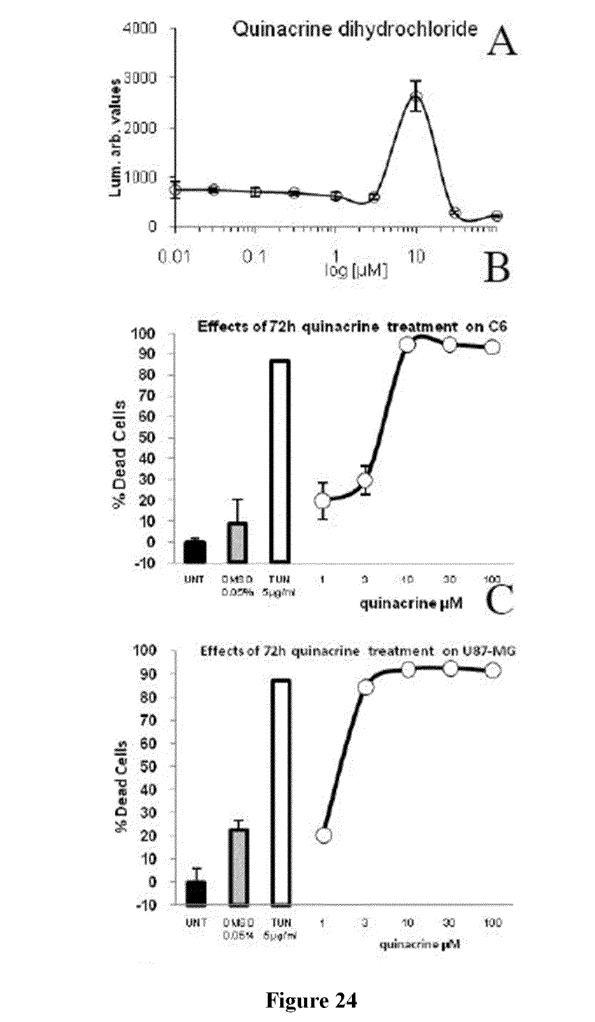

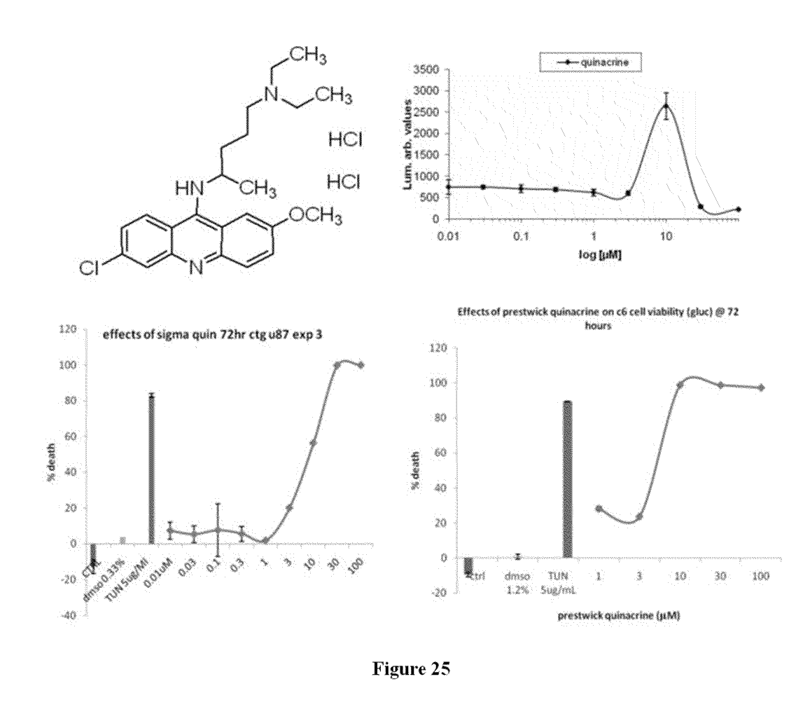

In FIG. 22 the graph was plotted in reference to the line of the negative control average+3 SD so that hits appear readily evident, as in the case of the single hitter in the figure. Based on the indications of the statistical analysis, the mini campaign detected 12 hits. Based on these data a hit rate of 1% or less was projected depending on how astringent hit selection was. In FIG. 23 the hits, defined as any value higher than the average of the controls+3 SD, were plotted as fold of induction above baseline and in reference to the threshold line. All the hit compounds were procured and are in the process of being followed up. Of the 12 hits, about 90% have been reproduced, when purchased from vendors and tested in a statistical manner in laboratory settings. Their EC₅₀ was established. The merits of the hits from a medicinal chemistry perspective were assessed through evaluation of structural features coupled with known biological activities of each of these hits. From such an evaluation, two compounds were initially selected as potentially attractive lead compounds (quinacrine and spiperone). Spiperone is a selective D2 dopamine receptor antagonist. This drug is used in the treatment of schizophrenia and other hallucinatory states. The second compound of choice is the drug quinacrine. In an effort to develop Structure-Activity Relationships around the chosen compounds, a search of commercially available compounds that are either close structural analogs of these lead compounds (for example, acridine and quinoline analogs of quinacrine) or are related to these in terms of their pharmacological activity (dopamine receptor antagonists related to spiperone) was performed. Seven additional D2 antagonists chemically related to spiperone were obtained. Three quinacrine analogs were obtained. In FIG. 24 the results obtained shows the effect of quinacrine on GRP78-Luc expression and gliotoxicity. Quinacrine increased expression of GRP78-luc up to 10 μM after which the toxic effect was preponderant. Parallely quinacrine in the same concentration range killed very effectively both C6 and U87-MG cells. In FIG. 25A, 25B and 25C the results related to the same experiments conducted with quinacrine are displayed. All effective compounds in the assay increased wild type GRP78 protein expression. Surprisingly, quinacrine is by far the most effective gliotoxic compound so far. The leads and SAR that emerges from the ongoing evaluations can be used for further lead optimizations.

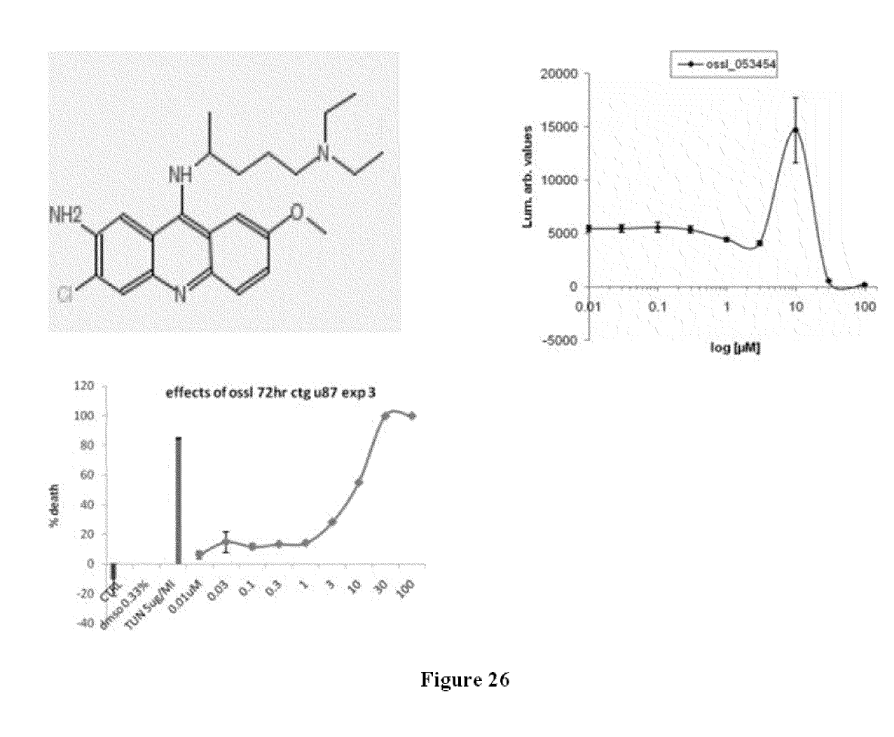

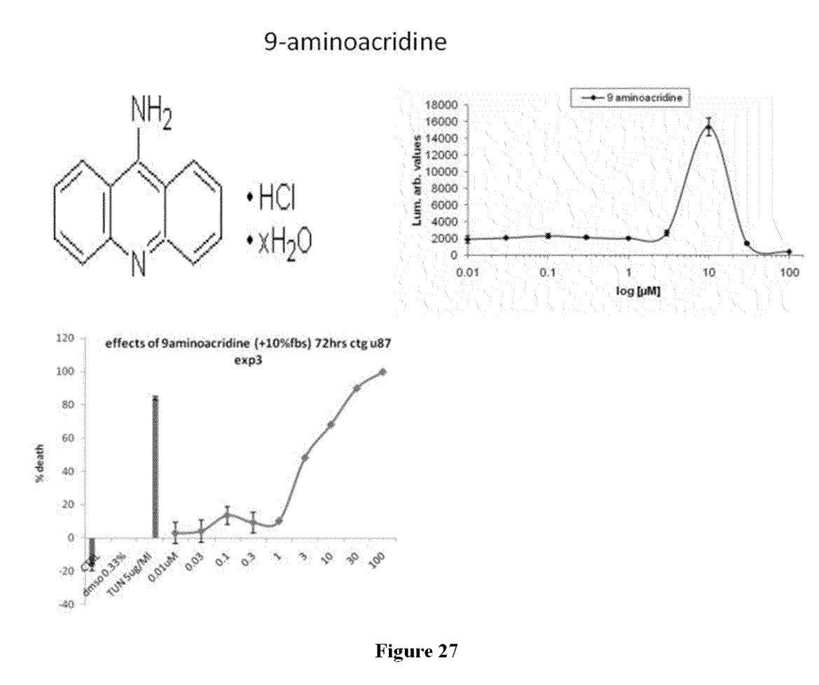

OSSL-053454 (3-chloro-N⁹-(5-(diethlyamino)pentan-2-yl)-7-methoxy acridine-2,9-diamine), a quinacrine analog, causes an increase of the GRP78-luc reporter gene similar to the increase caused by quinacrine (FIG. 26). Analogoulsy OSSL also causes gliotoxicity similar to that caused by quinacrine. Other quinacrine analogs such as 9-aminoacridine, hydrate, hydrochloride similarly causes increased expression of the reporter gene and also causes gliotoxicity (FIG. 27). Therefore these analogs are deemed to be effective quinacrine analogs.

Quinacrine shows a higher cytotoxicity in human cells than in rat cells. Chloroquine has been used in clinical trial in association with established anti-glioma medications for the treatment of gliomas (Briceno et al., 2003, “Therapy of glioblastoma multiforme improved by the antimutagenic chloroquine.” Neurosurg Focus 14(2): e3). Results from these trials are encouraging since patient survival was significantly prolonged (Briceno et al., 2003). What sets this study apart from these studies is that the authors were seeking the anti-mutagenic effect of Chloroquine, which is obtained at significantly lower concentrations of the agents. Previous studies also indicated that quinacrine was ineffective in killing C6 cells, however, again it was tested at less than 1 μM, for its antimutagenic effect, a concentration known to be inactive in C6 (Reyes et al., 2001, “Quinacrine enhances carmustine therapy of experimental rat glioma.” Neurosurgery 49(4): 969-73.). Properly adjusting the dose shows a significant effect of quinacrine in animal models of glioma.

Conclusive data has been provided indicating that changes in Ca²⁺ homeostasis reflect in abnormalities of Ca²⁺ storage in the ER. This in turns has allowed the identification ERSR abnormality in glioma cells. The data show, using two different activators of ERSR, non-correlated either chemically or by mechanisms of action, that ERSR is heightened in glioma cells as compared to normal astrocytes. This reflects in the induction of cell death in glioma cells to a larger extent than that observed in similar conditions in normal astrocytes. GRP78, an effector of ERSR, has been selected a bio sensor of ERSR activation. Data have been shown relative to the set up of an assay based on genetically engineered human glioma cells to express a GRP78p-LUC which allows identification of ERSR inducers. A hit rate of less than 1% has been projected and validation and S.A.R. on selected hits obtained with this miniscreen have been initiated. The preliminary data show a good correlation between the biosensor system and the final desired biological effect such as cytotoxicity.

### 6. Example 6

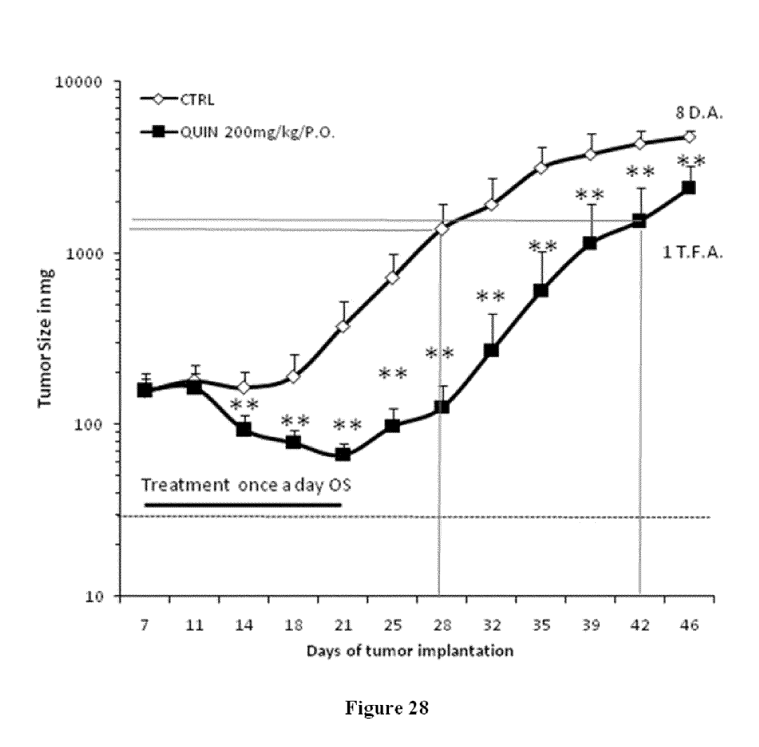

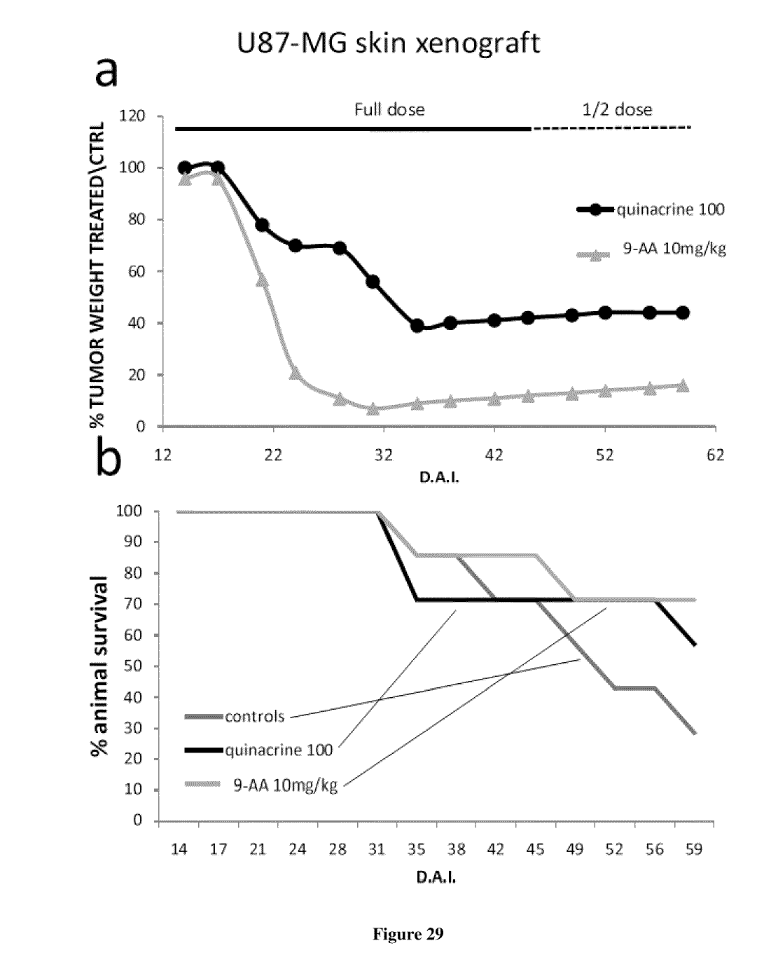

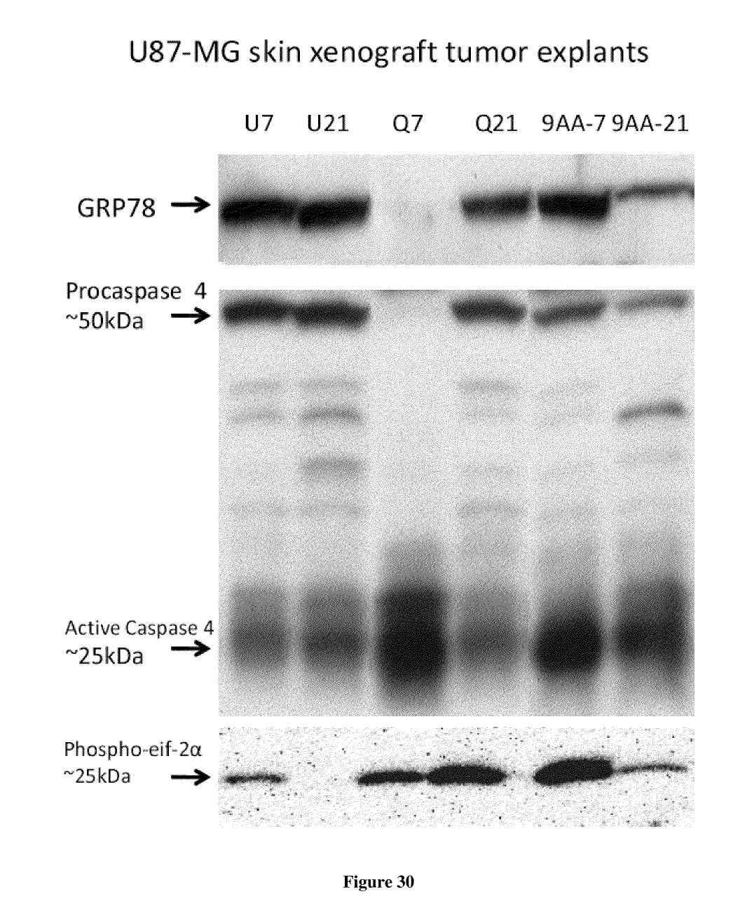

Mice bearing subcutaneous gliomas were received daily oral administration of 200 mg/kg of quinacrine. As shown in FIG. 28 Tumor growth was significantly inhibited by 6-7 folds at 21 days. The effect was already significant at 11 days. After withdrawal, the treated animals showed a significant reduction in tumor growth than controls. Animal studies showed a delay in tumor growth of 14 days which translates in 851 days in human and a 16% cure rate. In FIG. 29 is shown the effect of 100 mg/kg of quinacrine and 10 mg/kg 9-aminoacridine in Mice bearing subcutaneous U87-MG gliomas. It is evident the powerful antiglioma action of both compounds. Noticeably 9-aminoacridine is associated with 77% cure rate in these mice an effect never showed before.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of treating a subject with a glioma, comprising administering to the subject a therapeutically effective amount of an acridine analog or a pharmaceutically acceptable salt or hydrate thereof.

2. The method of claim 1, wherein the acridine analog has formula IA or IB: 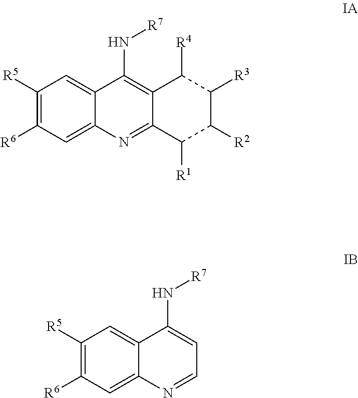
wherein the dashed line is either a single or double bond;
R¹, R², R³, and R⁴ are, independently of one another, hydrogen, amino, halide, hydroxy, optionally substituted alkyl, or optionally substituted alkoxy;
R⁵ represents hydrogen, amino, halide, hydroxy, methoxy, or ethoxy;
R⁶ represents hydrogen, halide, hydroxy, methoxy, or ethoxy; and
R⁷ represents a hydrogen, optionally substituted alkyl, or aminoalkyl, or a pharmaceutically acceptable salt or hydrate thereof.

3. The method of claim 2, wherein R⁷ is —CH(CH₃)(CH₂)₃NEt₂ or —CH(CH₃)(CH₂)₃N(Et)(EtOH).

4. The method of claim 1, wherein the acridine analog comprises 9-aminoacridine, 4-aminoquinoline, chloroquine, hydroxychloroquine, 3-chloro-N⁹-(5-(diethlyamino)pentan-2-yl)-7-methoxy acridine-2,9-diamine, or amodiaquine.

5. The method of claim 1, wherein the acridine analog comprises 9-aminoacridine.

6. The method of claim 1, wherein the acridine analog comprises quinacrine.

7. The method of claim 1, wherein the subject can be diagnosed with a need for treatment of a glioma.

8. The method of claim 1, further comprising the step of identifying a subject with a glioma.

9. A method of inhibiting intracranial metastasis of gliomal cancer cells in a subject, comprising administering to the subject an effective amount of an acridine analog or a pharmaceutically acceptable salt or hydrate thereof.

10. The method of claim 9, wherein the acridine analog has formula IA or IB: 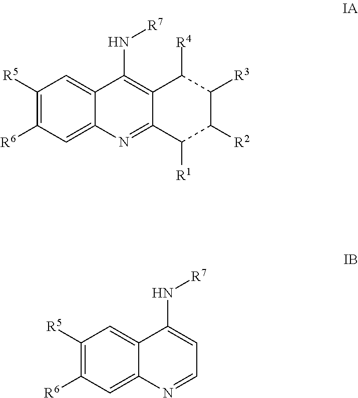
wherein the dashed line is either a single or double bond;
R¹, R², R³, and R⁴ are, independently of one another, hydrogen, amino, halide, hydroxy, optionally substituted alkyl, or optionally substituted alkoxy;
R⁵ represents hydrogen, halide, amino, hydroxy, methoxy, or ethoxy;
R⁶ represents hydrogen, halide, hydroxy, methoxy, or ethoxy; and
R⁷ represents a hydrogen, optionally substituted alkyl, or aminoalkyl, or a pharmaceutically acceptable salt or hydrate thereof.

11. The method of claim 10, wherein R⁷ is —CH(CH₃)(CH₂)₃NEt₂ or —CH(CH₃)(CH₂)₃N(Et)(EtOH).

12. The method of claim 9, wherein the acridine analog comprises 9-aminoacridine, 4-aminoquinoline, chloroquine, hydroxychloroquine, 3-chloro-N⁹-(5-(diethlyamino)pentan-2-yl)-7-methoxy acridine-2,9-diamine, or amodiaquine.

13. The method of claim 9, wherein the acridine analog comprises 9-aminoacridine.

14. The method of claim 9, wherein the acridine analog comprises quinacrine.

15. A method of preventing relapse in a subject previously treated for a glioma, the method comprising administering to the subject a prophylactically effective amount of an acridine analog or a pharmaceutically acceptable salt or hydrate thereof.

16. The method of claim 15, wherein the acridine analog has formula IA or IB: 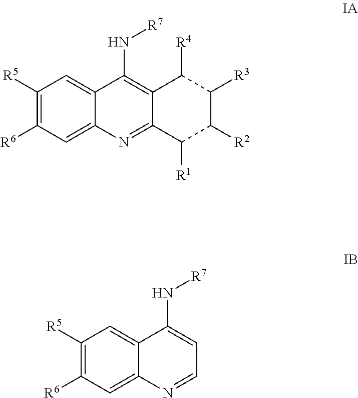
wherein the dashed line is either a single or double bond;
R¹, R², R³, and R⁴ are, independently of one another, hydrogen, amino, halide, hydroxy, optionally substituted alkyl, or optionally substituted alkoxy;
R⁵ represents hydrogen, amino, halide, hydroxy, methoxy, or ethoxy;
R⁶ represents hydrogen, halide, hydroxy, methoxy, or ethoxy; and
R⁷ represents a hydrogen, optionally substituted alkyl, or aminoalkyl, or a pharmaceutically acceptable salt or hydrate thereof.

17. The method of claim 16, wherein R⁷ is —CH(CH₃)(CH₂)₃NEt₂ or —CH(CH₃)(CH₂)₃N(Et)(EtOH).

18. The method of claim 15, wherein the acridine analog comprises 9-aminoacridine, 4-aminoquinoline, chloroquine, hydroxychloroquine, 3-chloro-N⁹-(5-(diethlyamino)pentan-2-yl)-7-methoxy acridine-2,9-diamine, or amodiaquine.

19. The method of claim 15, wherein the acridine analog comprises 9-aminoacridine.

20. The method of claim 15, wherein the acridine analog comprises quinacrine.
